(19) United States
(12) Patent Application Publication
(10) Pub. No.: US 2006/0135623 A1
(43) Pub. Date: Jun. 22, 2006

(54) Decrease in oxidative stress status through the administration of natural products and pharmaceutical drugs

(76) Inventor: Richard G. Cutler, Scottsdale, AZ (US)

Correspondence Address: MORRISON & FOERSTER LLP, 425 MARKET STREET, SAN FRANCISCO, CA, 94105-2482, US

(21) Appl. No.: 11/134,825

(22) Filed: May 20, 2005

Related U.S. Application Data
(60) Provisional application No. 60/573,106, filed on May 21, 2004.

(51) Int. Cl. A61K 31/12; C07C 49/20

(52) U.S. Cl. 514/675; 568/417

(57) ABSTRACT

The present invention provides compositions, methods, and kits for reducing oxidative stress thereby extending life span. The compositions, methods, and kits of the present invention can also be used be used to treat diseases of aging resulting from oxidative stress.

### CROSS-REFERENCE TO RELATED APPLICATION

This application claims priority to 60/573,106 filed on May 21, 2004, which is hereby incorporated by reference in its entirety.

### BACKGROUND

It was first suggested in 1956 that free radicals produced during aerobic respiration could cause cumulative oxidative damage, resulting in aging and death. The parallels between the effects of aging and of ionizing radiation, including mutagenesis, cancer, and gross cellular damage were noted. In particular, it had been discovered that radiolysis of water generated hydroxyl radicals which could be detected in living matter. This prompted investigators to hypothesize that endogenous oxygen radical generation occurred in vivo as a by-product of enzymatic redox chemistry. The enzymes involved in this process were presumed to be those involved in the direct utilization of molecular oxygen such as iron containing enzymes which catalyze oxidative reactions in vivo. In this manner, oxidative stress was postulated to be linked to aging, disease, and death. Specifically, a faster rate of respiration, with concurrent greater generation of oxygen radicals, would lead to more rapid aging and age associated diseases.

The free radical/oxidative stress theory of aging gained credibility with the discovery in 1969 of superoxide dismutase (SOD), an enzyme responsible for in vivo dismutation of O₂⁻ to H₂O₂. Later, organisms were found to possess additional antioxidant defenses. Among these defenses are enzymatic scavengers such as catalase and glutathione peroxidase which convert H₂O₂ to water, hydrophilic radical scavengers such as ascorbate, urate, and glutathione, lipophilic radical scavengers such as tocopherols, flavonoids, carotenoids, and ubiquinol, enzymes involved in the reduction of oxidized forms of small molecular antioxidants (GSH reductase, dehydroascorbate reductase) or responsible for the maintenance of protein thiols (thioredoxin reductase), and the cellular machinery that maintains a reducing environment (e.g., glucose-6-phosphate dehydrogenase, which regenerates NADPH).

Subsequently, the role of oxidative damage in the general decline in optimal bodily functions associated with the aging process has been appreciated. Furthermore, a number of diseases have been shown to be mediated by oxidative stress. Such oxidative stress mediated diseases include: Alzheimer's disease, autoimmune disease, cancer, cardiovascular disease, cataractogenesis, diabetes, iron overload, ischemic-reperfusion injury, macular degeneration, multiple sclerosis, muscular dystrophy, pancreatitis, Parkinson's disease, rheumatoid arthritis, and segmental progeria disorders.

A number of experimental studies in yeast and nematodes have shown that a substantial increase in lifespan, on the order of 20-30%, can be achieved by reducing oxidative stress. The decrease in oxidative stress status was achieved by methods such as dietary antioxidants, antioxidant mimetics, and caloric restriction or through the creation of transgenic animals having enhanced expression of specific antioxidant genes. These experimental treatments in model systems however are not practical in humans because of the severity of the treatments used. In particular, a 40% restriction in caloric intact was required in these studies, and the antioxidant mimetics were shown to have toxic side effects. Additionally, many antioxidant supplements are simply ineffective. As such, there is a tremendous need to provide a safe, non toxic pharmaceutical compound that can substantially protect individuals from oxidative stress, thus lengthening life span and reducing the incidence of age related diseases that result from oxidative damage.

### BRIEF SUMMARY

This invention provides a pharmaceutical composition that can substantially protect individuals from oxidative stress, thus lengthening life span and reducing the incidence of age related diseases that result from oxidative damage.

The present invention is directed to compositions containing geranyl geranyl acetone (GGA) analogues and to other compounds that have antioxidant activity.

The present invention is further directed to a method of extending life span in an individual by administering an effective amount of a GGA analogue to the individual to reduce oxidative stress and thereby extend the life span of the individual.

In the method of the invention, the mode of administering may be oral, gavage, intraperitoneal, or subcutaneous.

In one version of the method of the invention, the individual may be a vertebrate. In another version, the vertebrate is a human or a companion animal. The companion animal may be selected from dogs, cats, birds, rabbits, mice, hamsters, gerbils, ferrets, horses, fish, etc.

In another version, the present invention is directed to a method of treating an individual with a disease of aging resulting from oxidative stress by administering an effective amount of a GGA analogue to the individual.

The disease of aging may be chosen from Inflammatory/immune injury: glomerulonephritis, autoimmune diseases, rheumatoid arthritis, hepatitis, chronic inflammatory diseases; Ischaemia-reflow states: stroke, inflamed rheumatoid joint, ischaemia-reperfusion; Iron overload (tissue and plasma): idiopathic haemochromatosis, alcohol-related iron overload, cancer chemotherapy/radiotherapy Aging: disorders of premature ageing, ageing itself, age-related diseases, e.g. cancer; Red Blood Cells: lead poisoning, malaria, sickle cell anaemia; Respiratory tract: effects of cigarette smoke, emphysema, ARDS (Adult Respiratory Syndrome); Heart and cardiovascular system: atherosclerosis, cardiac iron overload, cardiac ischaemia-reoxygenation; Brain/nervous system/neuromuscular disorders: Alzheimer's disease, Parkinson's; Eye: cataract, retinopathy; Skin: UV radiation, contact dermatitis Cancer: Tumors, Carcinogenesis; Diabetes: hyperglycaemia, diabetic retinopathy, peripheral neuropathy, type II or non-insulin-dependent diabetes, juvenile onset and Cystic Fibrosis.

The present invention is further directed to a kit for treating an individual with a disease of aging resulting from oxidative stress. The kit may include a) a GGA analogue and b) means for applying the GGA analogue or related compound to a region of the body in need of treatment; and c) suitable packaging.

### BRIEF DESCRIPTION OF THE DRAWINGS

The patent or application file contains at least one drawing executed in color. Copies of this patent or patent application publication with color drawing(s) will be provided by the U.S. Patent and Trademark Office upon request and payment of the necessary fee.

FIG. 1 is a graph showing the effect of geranyl acetone (LSG 707) on life expectancy and lifespan in C. elegans.

FIG. 2 is a graph showing the effect of geranylgeranyl acetone (LSG 711) on life expectancy and lifespan in C. elegans.

FIG. 3 is a graph showing the extrapolation of life expectancy and lifespan extension using 500 μM geranylgeranyl acetone (LSG 711) in C. elegans to human survival.

FIG. 4 is a graph showing the effect of geranylgeranyl acetone (LSG 711) on lifespan in C. elegans.

FIG. 5 is a photomicrograph showing a reduction in the accumulation of lipofuscin pigment after geranylgeranyl acetone (LSG 711) treatment as visualized by confocal fluorescent microscopy.

FIG. 6 is a bar graph showing quantitatively the decrease in accumulation of lipofuscin pigment after treatment of C. elegans with various concentrations of geranylgeranyl acetone (LSG 711).

FIG. 7 is a graph showing the effect of 50 and 100 μM geranylgeranyl acetone (LSG 711) on cumulative percent loss of C. elegans over time.

FIG. 8 is a graph showing the effect of 500 μM geranylgeranyl acetone (LSG 711) on thermotolerance in C. elegans.

FIG. 9 is a bar graph showing an elevation of serum thioredoxin levels in human subjects after treatment with geranylgeranyl acetone (LSG 711).

FIG. 10 is a graph showing the effect of 50 and 100 μM of a geranylgeranyl acetone derivative (LSG 712) on lifespan in C. elegans.

FIG. 11 is a graph showing the effect of 50 and 100 μM of a geranylgeranyl acetone derivative (LSG 712) on cumulative percent loss of C. elegans over time.

FIG. 12 is a graph showing the effect of 100 μM of a geranylgeranyl acetone derivative (LSG 712) on thermotolerance in C. elegans.

### DETAILED DESCRIPTION

The inventor has identified certain compounds (referred to in this patent as “GGA analogues”) that may be used to treat certain diseases related to oxidative stress. In this Detailed Description are described the GGA analogues identified by the inventor, methods of making the GGA analogues, use of GGA analogues in the treatment or prevention of certain diseases, use of GGA analogues in combination with other compounds, formulations, and routes of administration and dosage forms for the GGA analogues.

Geranyl geranyl acetone analogues: Geranylgeranyl acetone (GGA) has the chemical formula:

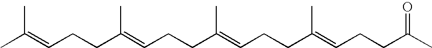

GGA analogs are GGA-like molecules with GGA antioxidant activity. Geranylgeranyl acetone has been demonstrated to have potent anti-ulcer activity and to induce heat-shock protein upregulation in rats and cell lines. Furthermore, the role of GGA as an antioxidant has been more clearly defined, as GGA has been shown to induce thioredoxin, a protein which plays an important protective role against oxidative stress. In particular, transgenic mice overexpressing thioredoxin were shown to have an increased lifespan. Based on these findings derivatives of GGA with varying numbers and configurations of double bonds and differing attached functional groups may also have potent antioxidant activity. Such analogues may derive their antioxidant activity from increasing the expression of thioredoxin or other genes involved in the protection of cells and tissues from oxidative stress. Non-limiting examples of such GGA analogues are shown below.

Class I: One class of GGA analogues (Class I) that may be used in the methods, formulations, dosages, combinations, and routes of administration described in this patent is compounds of the formula:

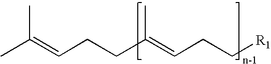

In an embodiment, Class I compounds can have n=8 or 9 and R1 can be any of the following functional groups: 1) acetone, 2) ethyl, 3) hydroxyalkyl 4) amine 5) hydroxyl 6) pivaloyl 7)formyl 8) propionyl 9) butryl) 10) valeryl 11) isovaleryl 12) stearoyl 13) myristoyl 14) palmitoyl 15) oleoyl 16) benzoyl 17) lauroyl.

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class II: Another class of GGA analogues (Class II) includes compounds of formula (I) in which n=8 or 9 and R1 can be any of the following functional groups: 1) 2-oxopropyl 2) 2-oxopentyl 3) 3-methyl-2-oxobutyl 4) amino 5) carbethoxyl amino 6) 2-hydroxypropyl 7) retinol acetate.

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class IIIa: Another class of GGA analogues (Class IIIa) includes derivatives of the geranyl geranyl family that have 5 instead of 4 double bonds with an additional double bond as illustrated below:

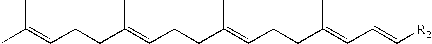

Class IIIa compounds can be in all-trans or a 5-cis form or can be a mixture of these two isomers. R₂ can be any one of the following functional groups: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) 2-hydroxypropyl.

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class IIIb: Another class of GGA analogues (Class IIIb) includes derivatives of the geranyl geranyl family that have 6 instead of 4 double bonds with additional double bonds as illustrated below:

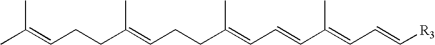

Class IIIb compounds can be in all-trans or a 5-cis form or can be a mixture of these two isomers. R₃ can be any one of the following functional groups: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) 2-hydroxypropyl.

These compounds may be synthesized using chemical synthesis methods familiar to those of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class IIIc: Another class of GGA analogues (Class IIIc) includes derivatives of the geranyl geranyl family that have 7 instead of 4 double bonds with additional double bonds as illustrated below:

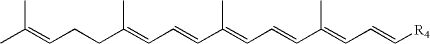

Class IIIc compounds can be in all-trans or a 5-cis form or can be a mixture of these two isomers. R₄ can be any one of the following functional groups: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) 2-hydroxypropyl.

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class IIId: Another class of GGA analogues (Class IIId) includes derivatives of the geranyl geranyl family that have 8 instead of 4 double bonds with additional double bonds as illustrated below:

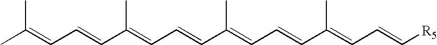

Class IIId compounds can be in all-trans or a 5-cis form or can be a mixture of these two isomers. R₅ can be any one of the following functional groups: 1) acetone, 2) ethyl, 3) hydroalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) 2-hydroxypropyl.

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class IVa: Another class of GGA analogues (Class IVa) includes derivatives of the geranyl geranyl family that have 3 instead of 4 double bonds, with the double bonds at positions C13 and C17 being saturated and with an additional double bond at the C I position as illustrated below:

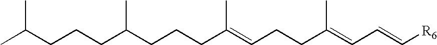

Class IVa compounds can be in all-trans or a 5-cis form or can be a mixture of these two isomers. R₆ can be any one of the following functional groups: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) 2-hydroxypropyl.

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class IVb: Another class of GGA analogues (Class IVb) includes derivatives of the geranyl geranyl family that have 2 instead of 4 double bonds with some double bonds being saturated and with a new double bond as illustrated below:

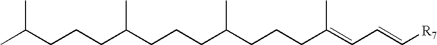

Class IVb compounds can be in all-trans or a 5-cis form or can be a mixture of these two isomers. R₇ can be any one of the following functional groups: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) 2-hydroxypropyl.

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class IVc: Another class of GGA analogues (Class IVc) includes derivatives of the geranyl geranyl family that have 1 instead of 4 double bonds with some double bonds saturated and with a single double bond as illustrated below:

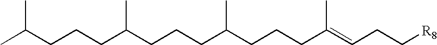

Class IVc compounds can be in all-trans or a 5-cis form or can be a mixture of these two isomers. R₈ can be any one of the following functional groups: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) 2-hydroxypropyl.

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class IVd: Another class of GGA analogues (Class IVd) includes derivatives of the geranyl geranyl family that have no double bonds as illustrated below:

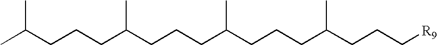

R₉ can be any one of the following functional groups: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) 2-hydroxypropyl.

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class V: Another class of GGA analogues (Class V) includes a 5-cis-geranylgeranyl compound as shown below:

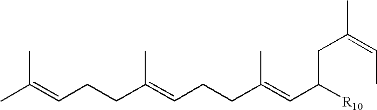

R₁₀ can be any one of the following functional groups: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) 2-hydroxypropyl.

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class VIa: Another class of GGA analogues (Class VIa) includes derivatives of α-carotene, as illustrated below, where n=2-4 and the isoprenoid units can exist in any reduced or form.

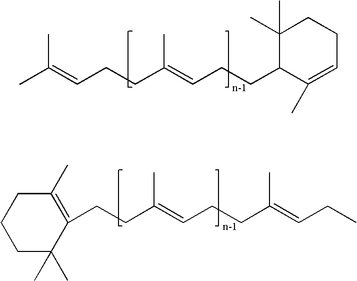

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class VIb: Another class of GGA analogues (Class VIb) includes derivatives of β-carotene, as illustrated below, where n=2-4 and the isoprenoid units can exist in any reduced or oxidized form.

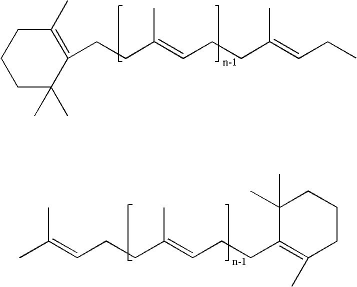

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class VIc: Another class of GGA analogues (Class VIc) includes derivatives of γ-carotene, as illustrated below, where n=2-4 and the isoprenoid units can exist in any reduced or oxidized form.

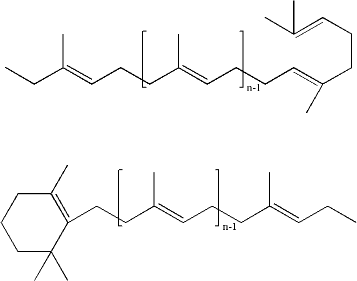

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class VId: Another class of GGA analogues (Class VId) includes derivatives of lycopene, as illustrated below, where n=24 and the isoprenoid units can exist in any reduced or oxidized form.

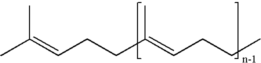

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class VIe: Another class of GGA analogues (Class VIe) includes derivatives of phytoene, as illustrated below, where n=2-6, 8, or 9 and the compound is either in a reduced or oxidized form.

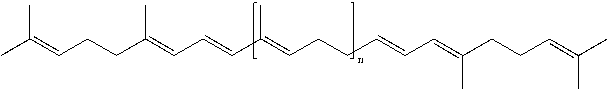

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class VIf: Another class of GGA analogues (Class VIf) includes derivatives of lutein, as illustrated below, where n=2-4 and the isoprenoid units can exist in any reduced or oxidized form.

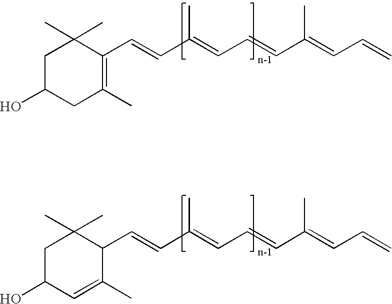

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class VIg: Another class of GGA analogues (Class VIg) includes derivatives of zeaxanthin, as illustrated below, where n=24 and the isoprenoid units can exist in any reduced or oxidized form.

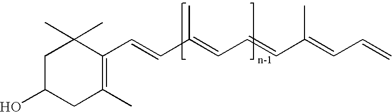

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class VIIa: Another class of GGA analogues (Class VIIa) includes derivatives of α-tocopherol (Vitamin E), as illustrated below, where n=24 and the isoprenoid units can exist in any reduced or oxidized form.

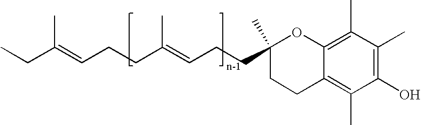

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class VIb: Another class of GGA analogues (Class VIb) includes derivatives of tocopherol, as illustrated below, where n=2-4 and the isoprenoid units can exist in any reduced or oxidized form.

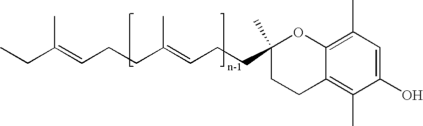

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class VIIc: Another class of GGA analogues (Class VIc) includes derivatives of δ-tocopherol, as illustrated below, where n=2-4 and the isoprenoid units can exist in any reduced or oxidized form.

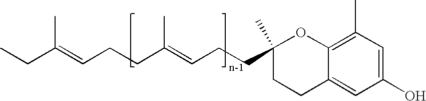

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class VIId: Another class of GGA analogues (Class VIId) includes derivatives of γ-tocopherol, as illustrated below, where n=2-4 and the isoprenoid units can exist in any reduced or oxidized form.

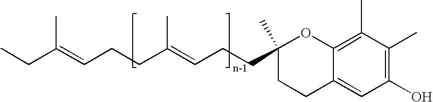

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class VIIIa: Another class of GGA analogues (Class VIIIa) includes derivatives of α-tocotrienol, as illustrated below, where n=1, 2, 4, 5, 6, 8, or 9, and the isoprenoid chain existing in any reduced or oxidized form.

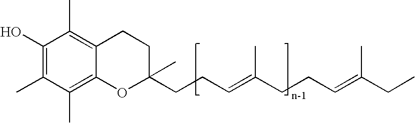

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class VIIIb: Another class of GGA analogues (Class VIIIb) includes derivatives of β-tocotrienol, as illustrated below, where n=1, 2, 4, 5, 6, 8, or 9, and the isoprenoid chain existing in any reduced or oxidized form.

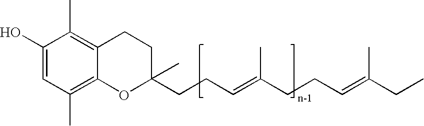

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class VIIIc: Another class of GGA analogues (Class VIIIc) includes derivatives of δ-tocotrienol, as illustrated below, where n=1, 2, 4, 5, 6, 8, or 9, and the isoprenoid chain existing in any reduced or oxidized form.

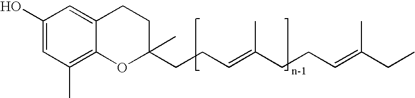

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class VIIId: Another class of GGA analogues (Class VIIId) includes derivatives of γ-tocotrienol, as illustrated below, where n=1, 2, 4, 5, 6, 8, or 9, and the isoprenoid chain existing in any reduced or oxidized form.

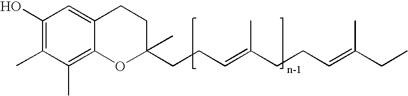

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class IXa: Another class of GGA analogues (Class IXa) includes derivatives of phylloquinone (Vitamin K₁), as illustrated below, where n=2-4, and the isoprenoid chain exists in any reduced or oxidized form.

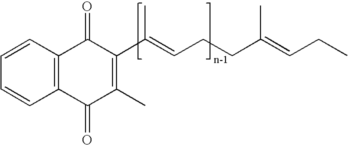

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class IXb: Another class of GGA analogues (Class IXb) includes derivatives of menaquinone (Vitamin K₂), as illustrated below, where n=2-4, and the isoprenoid chain exists in any reduced or oxidized form.

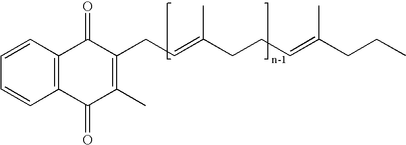

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class Xa: Another class of GGA analogues (Class Xa) includes derivatives of semiquinone (Coenzyme Q10) radical, as illustrated below, where n=2-6, 8, or 9.

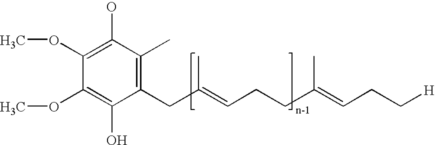

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class Xb: Another class of GGA analogues (Class Xb) includes derivatives of the reduced form of Coenzyme Q10, as illustrated below, where n=2-6, 8, or 9.

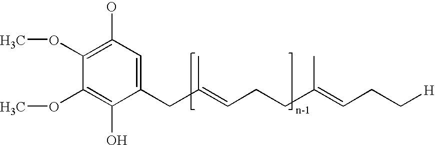

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class Xc: Another class of GGA analogues (Class Xc) includes derivatives of the oxidized form of Coenzyme Q10, as illustrated below, where n=2-6, 8, or 9.

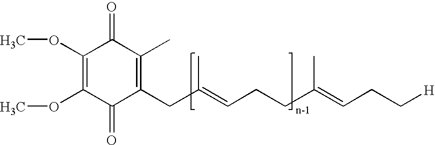

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class XIa: Another class of GGA analogues (Class XIa) includes derivatives of plaunotol, as illustrated below.

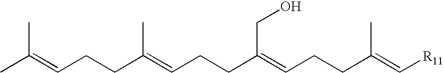

Class XIa compounds can be in all-trans or a 5-cis form or can be a mixture of these two isomers. R₁₁ can be any one of the following functional groups: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) 2-hydroxypropyl.

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class XIb: Another class of GGA analogues (Class XIb) includes derivatives of gefarnate, as illustrated below, where n=2-4, and the isoprenoid units can exist in any reduced or oxidized form.

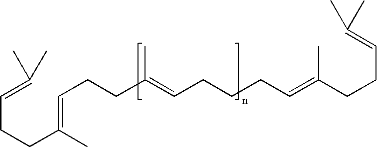

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class XII: Another class of GGA analogues (Class XII) includes derivatives of cholesterol glucoside, as illustrated below, where n=1-4, and the isoprenoid units can exist in any reduced or oxidized form.

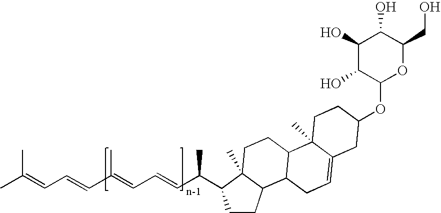

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class XIIIa: Another class of GGA analogues (Class XIIIa) includes derivatives of diferuloylmethane (curcumin), as illustrated below, where n=1-4, and the isoprenoid units can exist in any reduced or oxidized form.

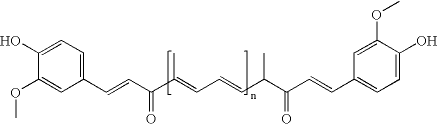

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class XIIIb: Another class of GGA analogues (Class XIIIb) includes derivatives of sulforaphane, as illustrated below, where n=1-4, and the isoprenoid units can exist in any reduced or oxidized form.

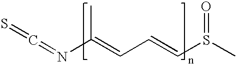

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class XIV: Another class of GGA analogues (Class XIV) includes derivatives of triterpenoid electrophiles, also known as avicins, as illustrated below. The R₁₂₋₁₅ groups of the Class XIV compositions shown below can be either hydrogen or a hydroxyl group.

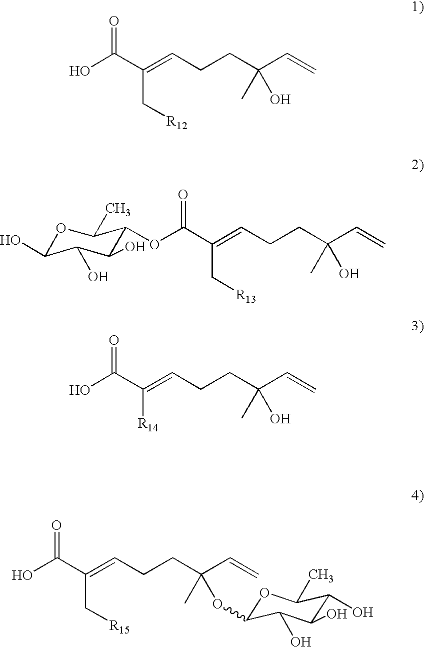

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class XVa: Another class of analogues (Class XVa) includes derivatives of docosahexaenoic acid, as illustrated below, where the structure may contain between 0-8 methyl groups.

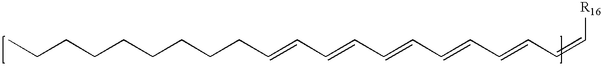

R₁₆ can be any one of the following functional groups: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) 2-hydroxypropyl.

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class XVb: Another class of analogues (Class XVb) includes derivatives of eicosapentaenoic acid, as illustrated below, where the structure may contain between 0-8 methyl groups.

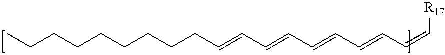

R₁₇ can be any one of the following functional groups: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) 2-hydroxypropyl.

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class XVc: Another class of analogues (Class XVc) includes derivatives of alpha linolenic acid, as illustrated below, where the structure may contain between 0-8 methyl groups.

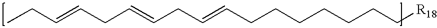

R₁₈ can be any one of the following functional groups: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) 2-hydroxypropyl.

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class XVIa: Another class of analogues (Class XVIa) includes derivatives of arachidonic acid, as illustrated below, where the structure may contain between 0-8 methyl groups.

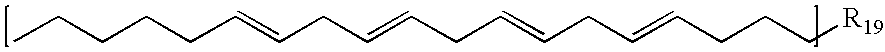

R₁₉ can be any one of the following functional groups: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) 2-hydroxypropyl.

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class XVIb: Another class of analogues (Class XVIb) includes derivatives of linoleic acid, as illustrated below, where the structure may contain between 0-8 methyl groups.

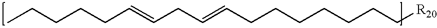

R₂₀ can be any one of the following functional groups: 1) acetone, 2) ethyl, 3) hydroxyalkyl 4) amine 5) hydroxyl 6)formyl 7) propionyl 8) butryl ) 9) 2-oxopropyl 10) 2-oxopentyl 1) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) 2-hydroxypropyl.

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class XVIc: Another class of analogues (Class XVIc) includes derivatives of gamma linolenic acid, as illustrated below, where the structure may contain between 0-8 methyl groups.

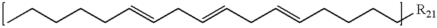

R₂₁ can be any one of the following functional groups: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) 2-hydroxypropyl.

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class XVIIa: Another class of analogues (Class XVIIa) includes derivatives of erucic acid, as illustrated below, where the structure may contain between 0-8 methyl groups.

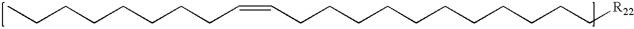

R₂₂ can be any one of the following functional groups: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) 2-hydroxypropyl.

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class XVIIb: Another class of analogues (Class XVIIb) includes derivatives of 11-docosenoic acid, as illustrated below, where the structure may contain between 0-8 methyl groups.

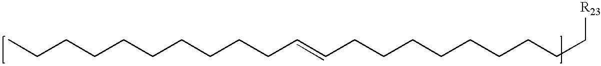

R₂₃ can be any one of the following functional groups: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) 2-hydroxypropyl.

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

Class XVIIc: Another class of analogues (Class XVIIc) includes derivatives of 5-docosenoic acid, as illustrated below, where the structure may contain between 0-8 methyl groups.

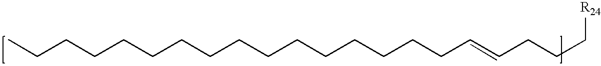

R₂₄ can be any one of the following functional groups: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) 2-hydroxypropyl.

These compounds may be synthesized using chemical synthesis methods familiar to one of ordinary skill in the art. General methods for chemical synthesis may be found in, among other sources, “Comprehensive Organic Transformations: A Guide to Functional Group Preparations”, Richard C. Larock, Wiley-VCH: 1999 and in “March's Advanced Organic Chemistry: Reactions, Mechanisms and Structure”, Jerry March & Michael Smith, John Wiley & Sons Inc.: 2001.

In the classes of compounds described herein, if a GGA analogue or related analogue contains stereochemistry, all enantiomeric and diastereomeric forms of the GGA analogues or related analogue are intended. Pure stereoisomers, mixtures of enantiomers and/or diastereomers, and mixtures of different GGA analogues or related analogue are described. Thus, the GGA analogues or related analogues may occur as racemates, racemic mixtures and as individual diastereomers, or enantiomers with all isomeric forms being included. A racemate or racemic mixture does not necessarily imply a 50:50 mixture of stereoisomers.

Where a given structural formula or chemical name is presented for a GGA analogue or related analogues, it is intended that all possible solvates, pharmaceutically acceptable salts, esters, amides, complexes, chelates, stereoisomers, geometric isomers, crystalline or amorphous forms, metabolites, metabolic precursors or prodrugs of the compound are also separately described by the chemical structural formula or chemical name.

Uses of GGA Analogues:

The GGA analogues described in this patent may be used to treat a variety of diseases including but not limited to diseases that result from oxidative stress.

Diseases resulting from oxidative stress: Diseases resulting from oxidative stress are those that result from acute or cumulative damage to macromolecules, cells, or tissues as a result of reactive oxygen species. A variety of diseases that result from oxidative stress can be treated with the compositions of this invention. Non-limiting examples of such diseases include those associated with aging such as: Inflammatory/immune injury^{1, 8, 9}: glomerulonephritis, autoimmune diseases, rheumatoid arthritis, hepatitis, chronic inflammatory diseases; Ischaemia-reflow states^{1, 7}: stroke, inflamed rheumatoid joint, ischaemia-reperfusion; Iron overload (tissue and plasma)¹: diopathic haemochromatosis, alcohol-related iron overload, cancer chemotherapy/radiotherapy; Aging^{1, 11, 12}: disorders of premature ageing, ageing itself, age-related diseases, e.g. cancer; red blood cells¹: lead poisoning, malaria, sickle cell anaemia; and Respiratory tract^{1, 10}: effects of cigarette smoke, emphysema, ARDS (Adult Respiratory Syndrome); Heart and cardiovascular system^{1, 2, 3, 4}: atherosclerosis, cardiac iron overload, cardiac ischaemia-reoxygenation; Brain/nervous system/neuromuscular disorders^{1, 14, 15, 16}: Alzheimer's disease, Parkinson's; Eye¹: cataract, retinopathy; Skin¹: UV radiation, contact dermatitis; Cancer^{1, 11, 12, 13}: tumors, carcinogenesis; Diabetes^{1, 5, 6}: hyperglycaemia, diabetic retinopathy, peripheral neuropathy, type II or non-insulin-dependent diabetes, juvenile onset; Cystic Fibrosis.^{1, 17} The footnotes above refer to the following references each of which are hereby incorporated by reference in their entirety.

Halliwell, B. and Gutteridge, J. M., 1999. Free radicals in biology and medicine, 3rd edn. Oxford University Press, New York. 2. Darley-Usmar, V. and Halliwell, B. (1996) Blood radicals. Pharm. Res. 13, 649. 3. Esterbauer, H. et al., (1992) The role of lipid peroxidation and antioxidants in oxidative modification of LDL. Free Rad. Biol. Med. 13, 341 4. Parthasarathy, S. et al., (1992) The role of oxidized LDL in the pathogenesis of atherosclerosis. Annu. Rev. Med. 43, 219 5. Hunt, J. V., (1995) Ascorbic acid and diabetes mellitus. In Subcellular Biochemistry, Vol. 25: Ascorbic Acid: Biochemistry and Biomedical Cell Biology (Harris, R J, ed.), p. 369. Plenum Press, New York. 6. Rosen, P. et al. (eds) (1998) Oxidative Stress and Antioxidants in Diabetes and its Complications, Marcel Dekker, USA. 7. Granger, D. N. and Kubes, P. (1994) The microcirculation and inflammation: modulation of leukocyte-endothelial cell adhesion. J. Leuk. Biol. 55, 6628. Stein, C. M. et al. (1996) Evidence of free radical-mediated injury (isoprostane overproduction) in scleroderma. Arth. Rheum. 39, 1146. 9. Lunec, J et al. (1994) 8OHdG. A marker of oxidative DNA damage in SLE. FEBS Lett. 348, 131. 10. Louie, S. et. al. (1996) ARDS: a radical perspective. Adv. Pharmacol. 38, 457. 11. Alberts, B. et al. (1996) Molecular Biology of the Cell, 3^{rd} edn. Garland Publishing, New York. 12. Elledge, S. J. (1996). Cell cycle checkpoints: preventing an identity crisis. Science 274, 1664. 13. Kensler, T. W. and Taffe, B. G. (1986) Free radicals in tumor promotion. Adv. Free Rad. Biol. Med. 2, 347. 14. Gotz, M. E. et. al. (1994) Oxidative stress: free radical production in neural degeneration. Pharmacol. Ther.63, 37. 15. Olanow, C. W. et al. (eds) (1996) Neurodegeneration and Neuroprotection in Parkinson's Disease. Academic Press, London. 16. Hsiao, K. et. al. (1996). Correlative memory deficits, A β elevation, and amyloid plaques in transgenic mice. Science 274, 99. 17. van der Vliet, A. et al. (1996) Oxidative stress in cystic fibrosis: does it occur and does it matter? Adv. Pharmacol. 38, 491.

In one method described in this patent, GGA and the GGA analogs described herein are used to increase lifespan and treat diseases related to oxidative stress. The GGA analogs described herein are also used to promote enhancement of health span. Enhancement of health span refers to an increase in general health and productivity at any age as an individual undergoes ageing. GGA has been shown to increase the expression of thioredoxin when administered to cells or animals. Furthermore, experimental animals overexpressing thioredoxin have been shown to have increased lifespan. GGA has also been shown to induce the expression of a heat shock protein, HSP70. An antioxidant response element (ARE) may be present in the regulatory elements of these genes. It is likely that GGA analogues will result in the expression of other genes that provide protection from oxidative stress. The inventor has shown that geranyl acetone and geranylgeranyl acetone increase lifespan and life expectancy in the model organism C. elegans. However, it was previously unknown whether GGA or GGA analogues would find use as a treatment for diseases resulting from oxidative stress. The inventor has further determined that the GGA analogues described in this patent may increase lifespan. This finding leads one of ordinary skill in the art to predict that some GGA analogues will reduce the incidence of age related diseases that result from oxidative stress and therefore will find use in treatment of diseases resulting from oxidative stress. However, given the vast number of possible analogues, it is not possible to predict which structures will be the most effective. The inventor has identified particular GGA analogues that could be especially effective in lifespan extension and which can be used in treating diseases resulting from oxidative stress.

Among the diseases that can be treated using the GGA analogues of the invention is Alzheimer's disease. Alzheimer's disease is a neurodegenerative disease characterized by neuronal cell death of groups of neurons. Alzheimer's afflicts a large percentage of the population. Alzheimer's disease afflicts about 4 million people in the United States, primarily the elderly, and is characterized by progressive memory loss, disorientation, depression and eventual loss of other body functions. The role of reactive oxygen species (ROS) and free radicals in the genesis and progression this disease has been increasingly appreciated in recent years. Among the suspected targets of oxidative damage in Alzheimer's disease are brain lipids. About 95 percent of the brain is made up of fatty acid lipids that, when attacked by free radicals, undergo peroxidation (oxidative damage.) This leads, in turn, to cell malfunction and eventual cell death. Thus, the GGA analogues of the present invention may be expected to serve as effective treatments for diseases such as Alzheimer's. The inventor has found that particularly useful GGA analogues for the treatment of Alzheimer's disease include one identified as class IIIa compound where R₂ is acetone (also designated herein as LSG 712). Other useful compounds may include a class IIIb compound where R₃ is acetone, a class IIIc compound where R₄ is acetone, and a class IIId compound where R₅ is acetone.

Another disease that may be treated using the GGA compounds of the present invention is diabetes. Recent studies have indicated that oxidative damage due to hyperglycemia contributes to the microvascular pathology of diabetes that occurs particularly in the retina, renal glomerulus, and peripheral nerves, causing blindness, renal failure, and peripheral neuropathy. Furthermore, although the death of beta-cells that underlies type 1 diabetes is probably due to an autoimmune response, the particular susceptibility of beta-cells to oxidative damage from reactive oxygen species (ROS) produced during inflammation may be a predisposing factor. The association between hyperglycemia and oxidative damage has been noted for some time with various sources proposed for the underlying ROS. Recently, it has been suggested that increased mitochondrial ROS production during hyperglycemia may be central to much of the pathology of diabetes. Thus, mitochondrial ROS production and oxidative damage may contribute to the onset, progression, and pathological consequences of both type 1 and type 2 diabetes. For these reasons, the GGA compounds, including GGA analogs, and in particular, the class IIIa analogs, including LSG 712, may be useful treatments for diabetes as well. Other useful compounds may include a class IIIb compound where R₃ is acetone, a class IIIc compound where R₄ is acetone, and a class IIId compound where R₅ is acetone.

Extension of life span is meant to include extension of life beyond the average life span of an individual. Such an extension of life span can be preferably an extension of 5 %; even more preferably it can be an extension of 10% or more. Enhancement of health span refers to an increase in general health and productivity at any age as an individual undergoes ageing.

The effect of GGA analogues on extension of life span can be determined using as an assay system, the nematode, C. elegans, an organism widely used for studies on aging. GGA analogues can be fed to nematodes and the rate of accumulation of lipofuscin pigment can be monitored using fluorescent microscopy. Lipofuscin is generally thought to be a product of oxidized lipids/proteins that accumulate with age in a remarkably linear fashion in all animals. Lipofuscin is often used as a biomarker of aging in many species including nematodes, Drosophila, mice, and primates. The inventor has discovered that treatment of nematodes with GGA reduces the age-related accumulation of lipofuscin pigment.

The extension of life span in the nematode can also be directly measured. The inventor has discovered that feeding GGA to nematodes increased both the mean and maximum lifespan of the nematode by about 50 percent. The increase in lifespan found in the nematode is equivalent to an increase in human life expectancy from 75 years to about 112 years and human lifespan from 120 years to 180 years.

Additionally, the nematode system can be used to determine the effect of GGA analogues on the induction of thioredoxin and heat shock protein (HSP) 70 expression, on the reduction of general oxidative stress, and on enhancement of general activity levels, vitality, and vigor. Microarray technology can be used to perform genome wide gene expression profiles to determine how GGA analogues exert their actions and thus suggest other targets that may be important in life extension. These studies on nematodes can be extended to mammals including mice, dogs, and humans.

GGA and other acyclic polyisoprenoid compounds have been shown to be effective in treating gastric ulcers in rats. See Murakami et al. (1983) Japan. J. Pharmacol. 33, 549-556. GGA has also been shown to have antioxidant activities. Among the other health related effects of GGA and related compounds are: protection against ethanol induced apoptotic DNA fragmentation in mucosal cells (Mizushima et al. (1999) Dig. Dis. Sci. 44, 510-514), induction of differentiation of various human myeloid leukemia cell lines (Sakai et al. (1993) Biochem. Biophys. Res. Commun. 191, 873-879), prevention of primary nonfunction in rat liver transplantation (Fudaba et al. (1999) Transplant Proc. 31, 2918-2919; Fudaba et al. (2000) Transplant Proc. 32, 1615-1616; Fudaba et al. (2001) Transplantation 72, 184-189), protection against ischemia/reperfusion injury in rat heart (Ooie et al. (2001) Circulation 104, 1837-1843; Yamanaka (2003) J. Mol. Cell Cardiol. 35, 785-794), induction of antiviral gene expression in human hepatoma cells (Ichikawa et al. (2001) Biochem. Biophys. Res. Commun. 280, 933-939), protection of human monocytes from mitochondrial membrane depolarization (Aron et al. (2001) Cell Mol. Life Sci. 58, 1522-1527), suppression of inflammatory responses and improvement of survival after massive hepatectomy in rats (Oda et al. (2002) J. Gastrointest. Surg. 6, 464472; discussion p. 473), reduction in cellular damage induced by proteasome inhibition in cultured spinal neurons (Kikuchi et al. (2002) J. Neurosci Res 69, 373-381), protection of retinal ganglion in a rat glaucoma model (Ishii et al. (2003) Invest. Ophthalmol. Vis. Sci. 44, 1982-1992), protection of mucous cells against toxic oxygen metabolites (Hiraishi et al. J. Lab. Clin. Med. 121, 570-578), stimulation of mucin synthesis by induction of neuronal nitric oxide synthase (Rokutan et al. (2000) J. Gastroenterol. 35, 673-681), protection against aspirin-induced changes in gastric glycoproteins (Oketani et al. Jpn. J. Pharmacol. 33, 593-601). The compounds described in this patent may have similar utilities based on similar chemical features. The inventor has shown that administration of GGA to humans has been shown to increase serum levels of thioredoxin after ingestion of this compound.

As a first step in the administration of GGA analogue compositions, individuals must be first selected for treatment with the compositions and methods of the invention by virtue of being in need of increasing their lifespan, being in need of oxidative stress reduction or by virtue of suffering from a disease of aging resulting from oxidative stress. The individual can be selected after diagnosis by a health care provider as requiring an increase in lifespan, or as suffering from oxidative stress or a disease of aging resulting from oxidative stress.

The individual so selected may be a vertebrate. In an embodiment, the individual may be a mammal. In another embodiment, the individual may be an experimental animal. In another embodiment, the individual may be a human. In yet another embodiment, the individual maybe a companion animal. A companion animal is one which is kept as a pet and may be, for example, a dog, cat, rodent, primate, rabbit, birds or horse.

GGA analogues in combination with other compounds: The present invention provides a pharmaceutical composition including a therapeutically effective amount of a GGA analogue. The GGA analogues of this invention can be provided singly or in combinations of two or more. Alternatively, one or more additional agents that reduce oxidative stress can be combined with the GGA analogues of this invention. Such additional agents may include vitamin C, β-carotene, α-tocopherol, β-CATECHIN, N-acetyl-cysteine, and N-tert-butyl-α-phenylnitrone. Furthermore, the GGA analogues of this invention can be combined with agents such as statins. These compositions can be contained within pharmaceutically acceptable carriers and/or diluents.

Formulations, Routes of Administration, and Dosage Forms:

The active ingredients of a pharmaceutical composition comprising GGA analogues are contemplated to exhibit therapeutic activity when administered in an amount which depends on the particular application. The dosage regime may be adjusted to provide the optimum therapeutic response. For example, several divided doses may be administered daily or the dose may be proportionally reduced as indicated by the exigencies of the therapeutic situation. The active compound may be administered in a convenient manner such as by the oral, intravenous (where water soluble), intramuscular, subcutaneous, intranasal; intradermal or suppository routes or implanting (e.g., using slow release molecules). Depending on the route of administration, the active ingredients which comprise the pharmaceutical composition of the invention may be required to be coated in a material to protect the ingredients from the action of enzymes, acids and other natural conditions which may inactivate said ingredients.

The active compounds may also be administered parenterally or intraperitoneally. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders of the extemporaneous dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When GGA analogues and additional agents are suitably protected as described above, the active composition may be orally administered, for example, with an inert diluent or with an assimilable edible carrier, or it may be enclosed in hard or soft shell gelatin capsule, or it may be compressed into tablets, or it may be incorporated directly with food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be carried and may conveniently be between about 5 to 80% of the weight of the unit. The amount of active compound in such therapeutically useful compositions is such that a suitable dosage will be obtained. The tablets, troches, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor of course, any material used in preparing any dosage unit form should be pharmaceutically pure and substantially non-toxic in the amounts employed. In addition, the active compound may be incorporated into sustained-release preparations and formulations.

The GGA analogue compositions of the present invention are administered in a therapeutically effective amount, which is understood to mean a nontoxic but sufficient amount of the drug or agent to provide the desired effect. For example, an effective amount means the amount that results in improvement in lifespan or that results in improvement or prevention against an age related disease that results from oxidative stress.

As used herein “pharmaceutically acceptable carrier and/or diluent” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the GGA analogues, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the individuals to be treated; each unit containing a predetermined quantity of active material calculated to produce the desired therapeutic effect in association with the required pharmaceutical carrier. The specification for the dosage unit forms of the invention are dictated by and directly dependent on (1) the characteristics of the GGA analogues and the particular therapeutic effect to be achieved, and (b) the limitations inherent in the art of compounding.

A preferred formulation for GGA analogues is in an oil. Suitable oils for this type of formulation are well known in the art and can include vegetable oils or mineral oil. The oil containing GGA analogs can also be absorbed to a solid matrix using methods known in the art. Given the potential susceptibility of GGA analogs to oxidation, and in particular, peroxidation of double bonds, formulations of GGA analogs will preferably contain an antioxidant or other preservative or stabilizing compounds. A preferred preservative is vitamin E.

Kits: The present invention also provides kits to administer the compositions of the invention to the body in particular, to specialized areas of the body that require treatment. For example, the kits can include components for administration of the compositions to areas of the body affected by damage resulting from oxidative stress. The kits are characterized as containing: (a) a means for containing a therapeutic composition including a GGA analogue and optionally an additional oxidative stress reducing agent and (b) a means for administering the compound or composition to the appropriate region of an individual. When the composition is in the form of a suppository, the means for containing the compound or composition may be foil or plastic wrappers surrounding the suppositories that may be placed into a box or carton or other sealed container. The means for containing the compound or composition may be a bottle, canister or plastic tube when the composition is in the form of a liquid, gel, lotion or cream. Rings or catheters containing the compositions may be placed in individual foil or plastic wrappers and then placed into a box or carton. The means for administering the compound or composition may be a catheter, a medicated ring, suppository, dropper, syringe, applicator, tube or by spray. When the composition is a liquid, the administration may be accomplished by means of a dropper, syringe, catheter or finger tip. When the composition is in the form of a gel, lotion, or cream the administration may be carried out by means of a tube, dropper, syringe, catheter or finger tip.

The kits of this invention will also typically include means for packaging the container means and the administering means. Such packaging may take the form of a cardboard or paper box, a plastic or foil pouch, etc. The present kits will also usually include written instructions that describe how to administer the therapeutic compound or pharmaceutical composition containing the therapeutic compound to the region of the body in need of treatment. It is to be understood that the written instructions may be on any of the container means, the administering means, or the packaging means, in addition to being present on a separate piece of paper.

The invention will be better understood by reference to the following non-limiting examples.

### EXAMPLE 1

The effect of geranylacetone (GA) and geranylgeranyl acetone (GGA) on the lengthening of lifespan and life expectancy was studied using the model organism, C. elegans, a system that is commonly used to study aging. GA and GGA were tested at three different concentrations of 5 μM 50 μM and 500 μM. The drugs were dissolved in ethanol, and thus ethanol with no drug present was used as the control in these experiments. 100 worms were used to test each concentration of the drug. GA is assumed to be a pure drug at 4.3 M and was dissolved in 100% ethanol. The drug was then diluted in S basal plus cholesterol plus bacteria OP50 such that the final concentration of ethanol on all plates was 1%. GGA was treated as 10% active with an inert carrier. Thus, all treatments were done with a 10-fold increase over the calculated concentration. GGA was suspended in 100% ethanol at 50 degrees Celsius and mixed for about hour after which the supernatant from this mixture was used. The drug was then diluted in S basal plus cholesterol plus bacteria OP50 such that the final concentration of ethanol on all plates was 1%.

Wild type N2 C. elegans were obtained from the University of Minnesota Caenorhabditis Genetics Center. All assays were conducted on OP50 (CGCb) using 1× M9 OP50 worm food solution. Survival experiments were carried out in duplicate culture (2 cohorts, 50 worms each) at 25 degrees Celsius. Worms were exposed to the drugs at day 3 of life when set up in liquid culture with OP50 at 10⁹ bacteria/ml. Worms were transferred daily for the first six days to remove progeny and to assess mortality. At day 10 and thereafter transfers were done 3 times per week. At each transfer, fresh drug was added to the desired concentration in a manner to achieve a final ethanol concentration of 1%.

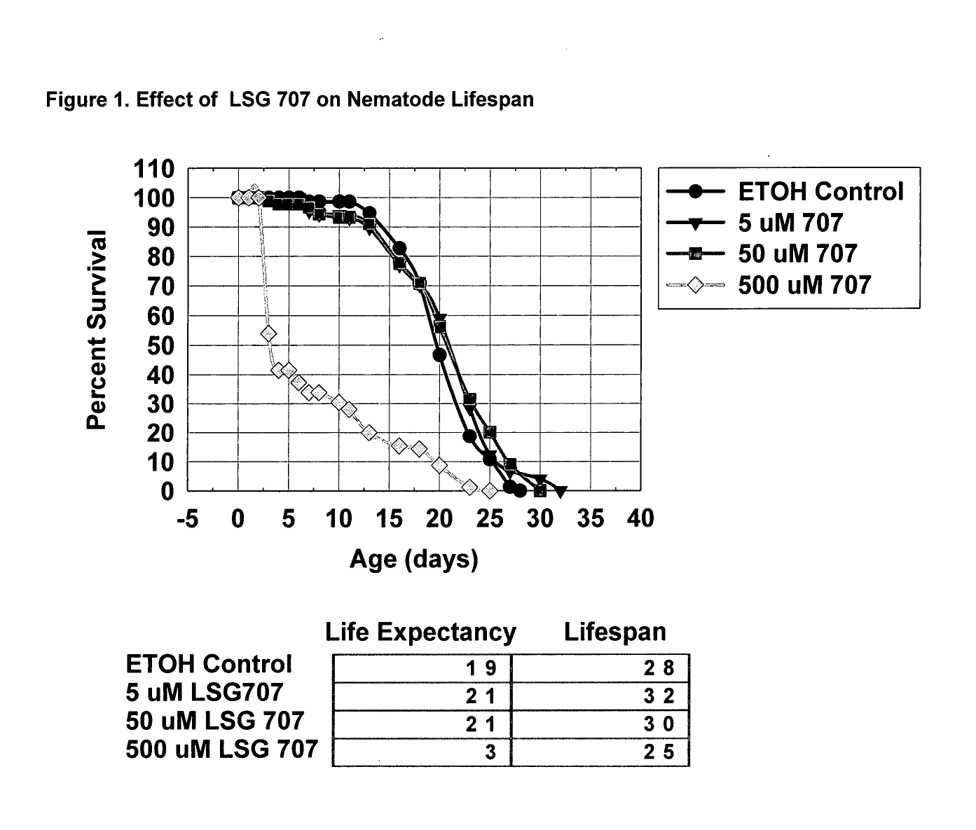

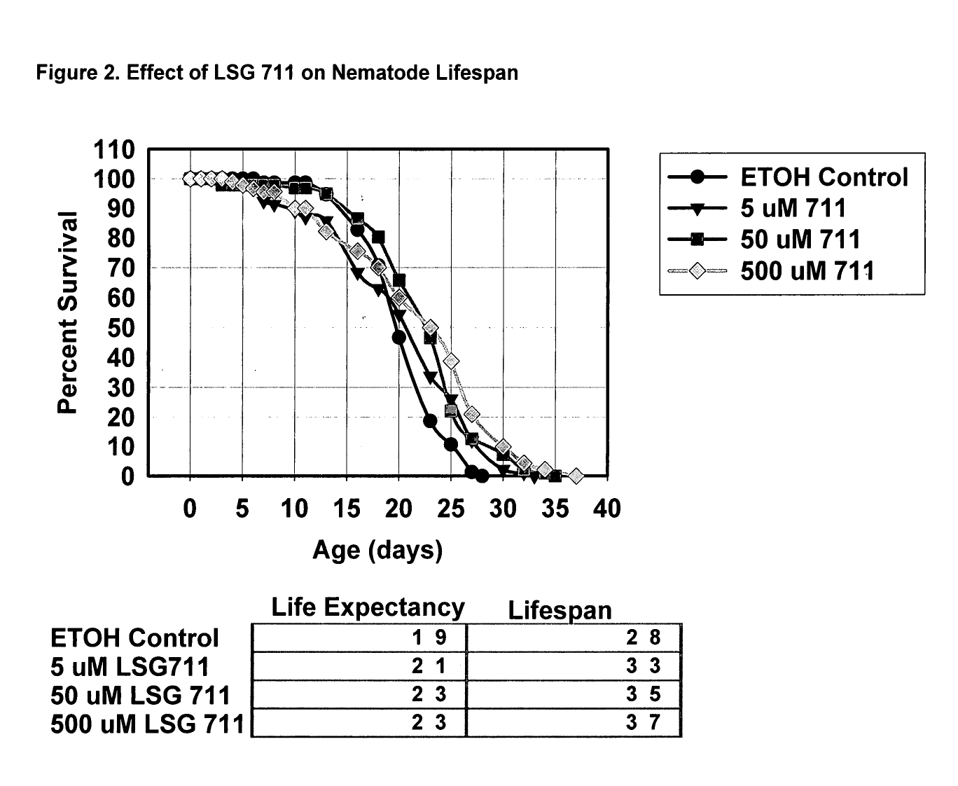

The results of these experiments are shown in FIGS. 1 and 2. FIG. 1 shows that both 5 and 50 μM GA (denoted LSG 707 in the figures) increases life expectancy from 19 to 21 days. Lifespan is increased from 28 to 32 and 30 days when 5 and 50 μM GA respectively was used. FIG. 2 shows that GGA (denoted LSG 711 in the figures) increases life expectancy from 19 to 21, 23, and 23 days when 5, 50, and 500 μM GGA respectively was used. Lifespan is increased from 28 to 33, 35, and 37 days when 5, 50, and 500 μM GGA respectively was used.

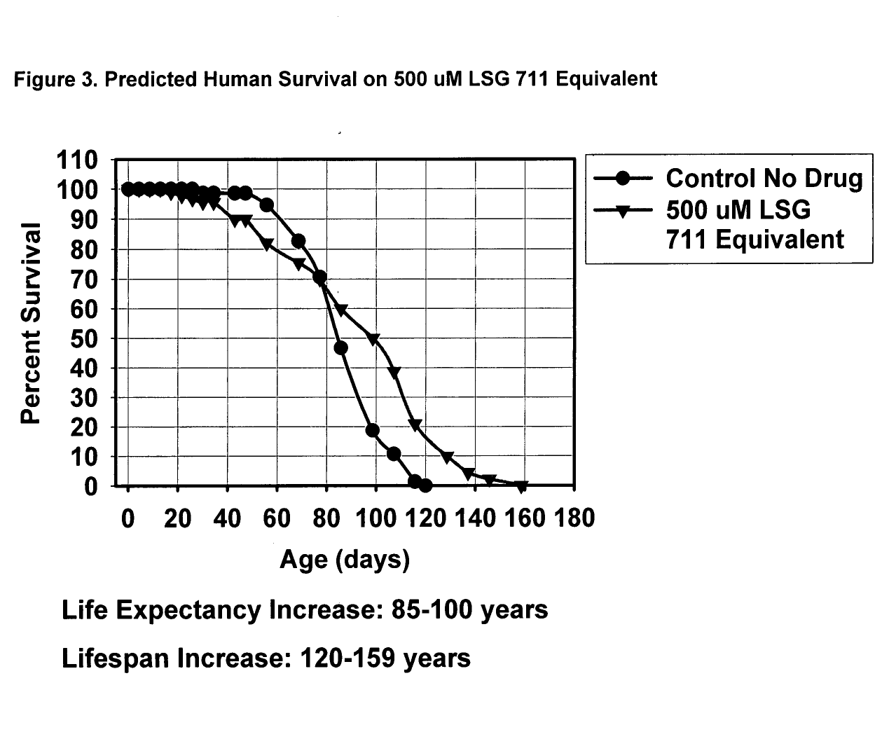

Extrapolation of the survival data from the C. elegans studies to human life expectancy and lifespan is shown in FIG. 3. FIG. 3 shows that 500 μM GGA is predicted to increase life expectancy to 85-100 years and to increase life span to 120-159 years.

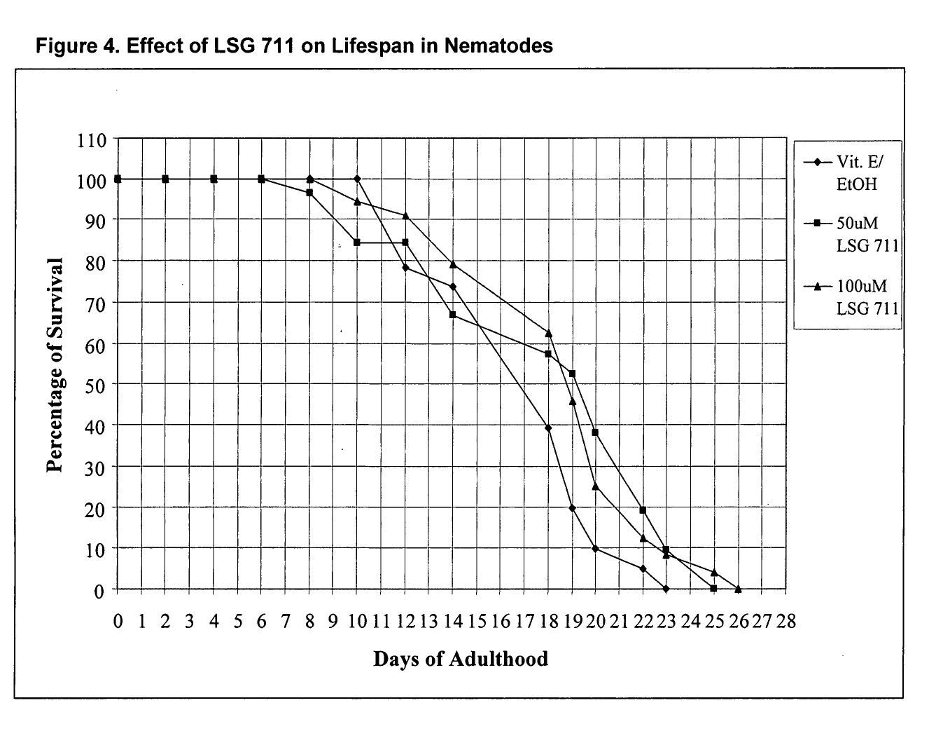

Additional studies to determine the effect of GGA on lifespan were performed using agar culture conditions. The results of these studies using 50 and 100 μM GGA are shown in FIG. 4. The data in FIG. 4 shows that 50 μM GGA produces an 11% mean and 8% maximum percent increase in lifespan (Pᵥₐₗᵤₑ=0.1465). 100 μM GGA produces an 11% mean and 13% maximum percent increase in lifespan (Pᵥₐₗᵤₑ=0.039).

### EXAMPLE 2

The effect of GGA on the age dependent accumulation of lipofuscin pigment was measured in C. elegans after treatment of worms with various concentrations of the drug. Wild type C. elegans were obtained from the University of Minnesota Caenorhabditis Genetics Center. A 250 mM stock of GGA was dissolved in ethanol and then diluted into 1× M9 OP50 worm food solution. Synchronized eggs were placed on plates containing no drug, 5 μM, 50 μM, or 500 μM GGA and allowed to hatch. On day 8, the worms were washed in 9M buffer and placed on a microscope slide. The amount of lipofuscin pigment was measured using a confocal microscope measuring fluorescence at 488 nm. Fluorescent scans were performed to quantify the amount of lipofuscin pigment. The quantification was normalized to the total area size of the worm in nm².

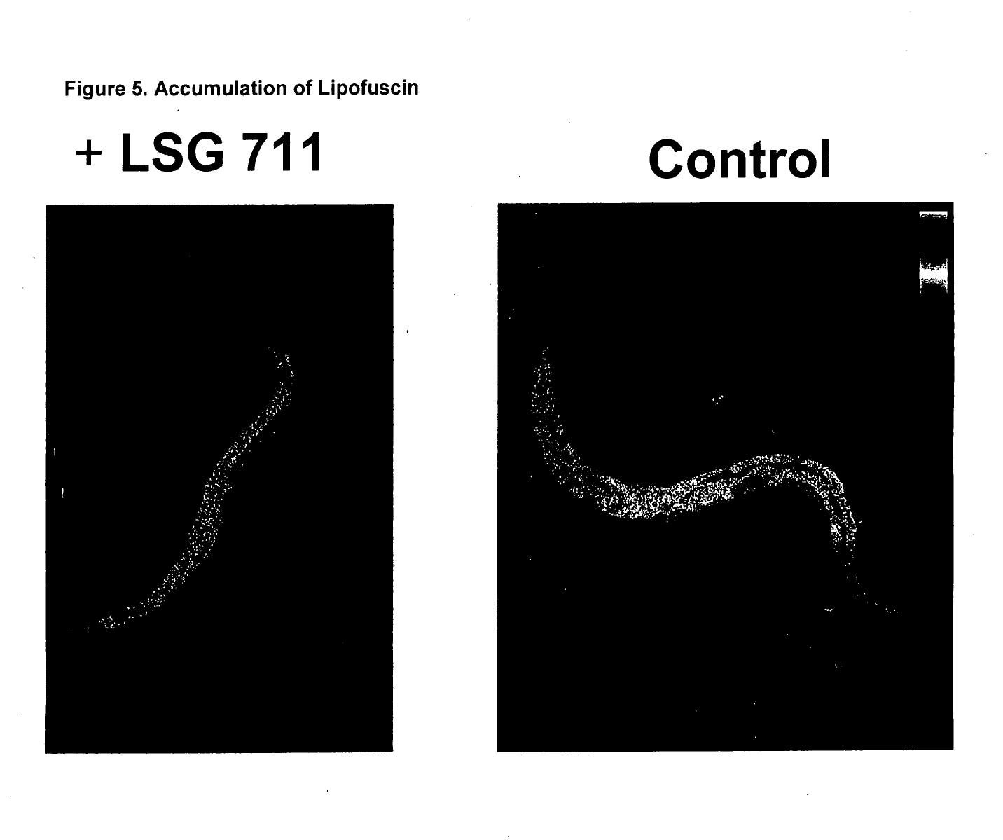

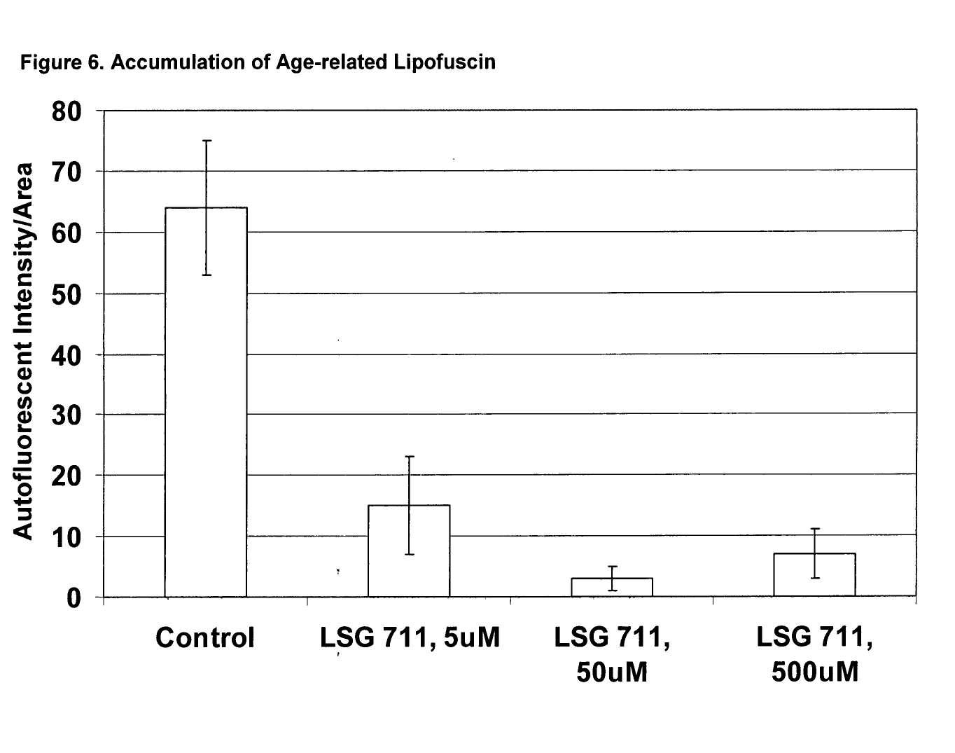

FIG. 5 shows that treatment of C. elegans with GGA results in a reduction of lipofuscin accumulation as determined by cofocal fluorescence microscopy. FIG. 6 shows that treatment of worms with GGA substantially reduces the accumulation of lipofuscin from a control value of about 65 autofluorescent intensity/area units to about 15, under 5, and under 10 autofluorescent intensity/area units when 5, 50, and 500 μM GGA, respectively, was used.

### EXAMPLE 3

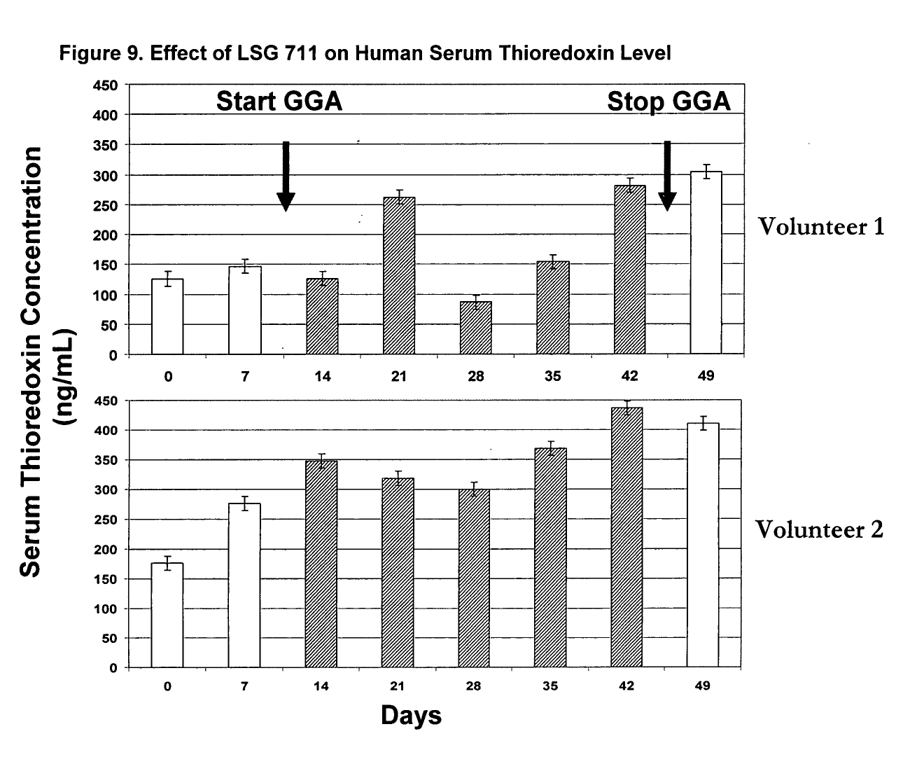

To test the effect of GGA in providing oxidative stress protection in humans, 2 human volunteers were given three 50 mg capsules of GGA per day. Serum levels of thioredoxin, a protein important in protection from oxidative stress was measured at various time intervals as shown in FIG. 9. As shown in FIG. 9 after approximately 32 days of receiving GGA, one volunteer showed an increase of serum thioredoxin to about 275 ng/ml from a pre-treatment level of about 175 ng/ml. A second volunteer showed an increase of serum thioredoxin to about 425 ng/ml from a pre-treatment level of about 175 ng/ml.

### EXAMPLE 4

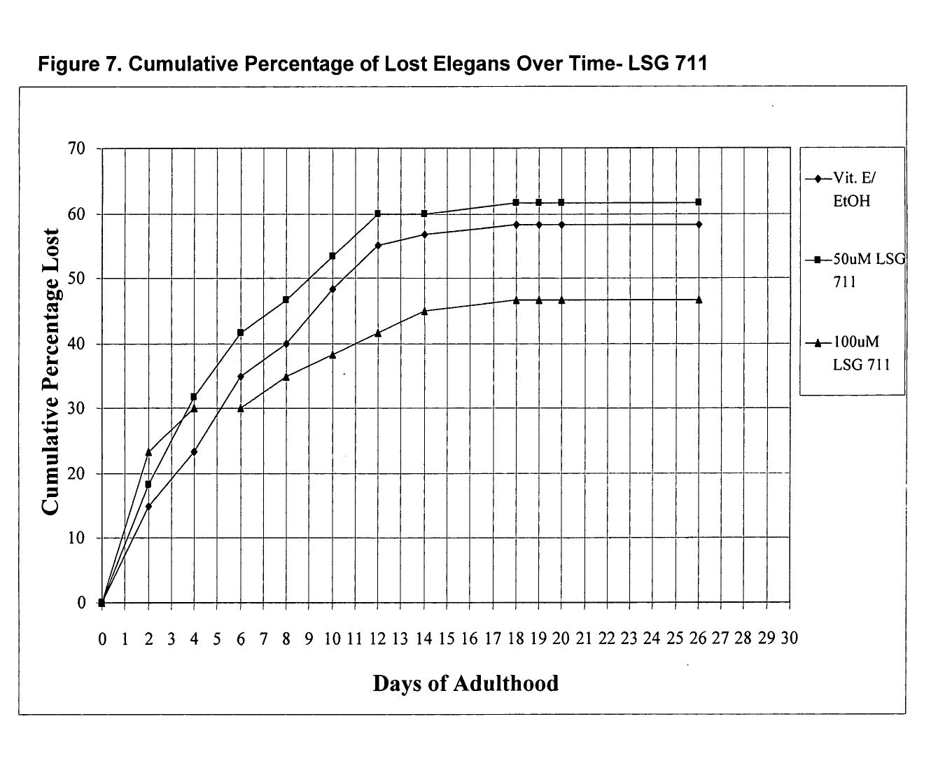

Another assay that can be used to measure lifespan extension in C. elegans is to measure the cumulative percent loss of C. elegans cultured on agar plates over time in the presence of GGA (LSG 711). C. elegans were maintained as described above. The results of using 50 and 100 μM GGA in this type of assay are shown in FIG. 7. The use of 50 μM GGA resulted in a −7% maximum and −4% mean percent increase in protection (Pᵥₐₗᵤₑ<0.0001). The use of 100 μM GGA resulted in a 12% maximum and 7% mean percent increase in protection (Pᵥₐₗᵤₑ<0.0001).

### EXAMPLE 5

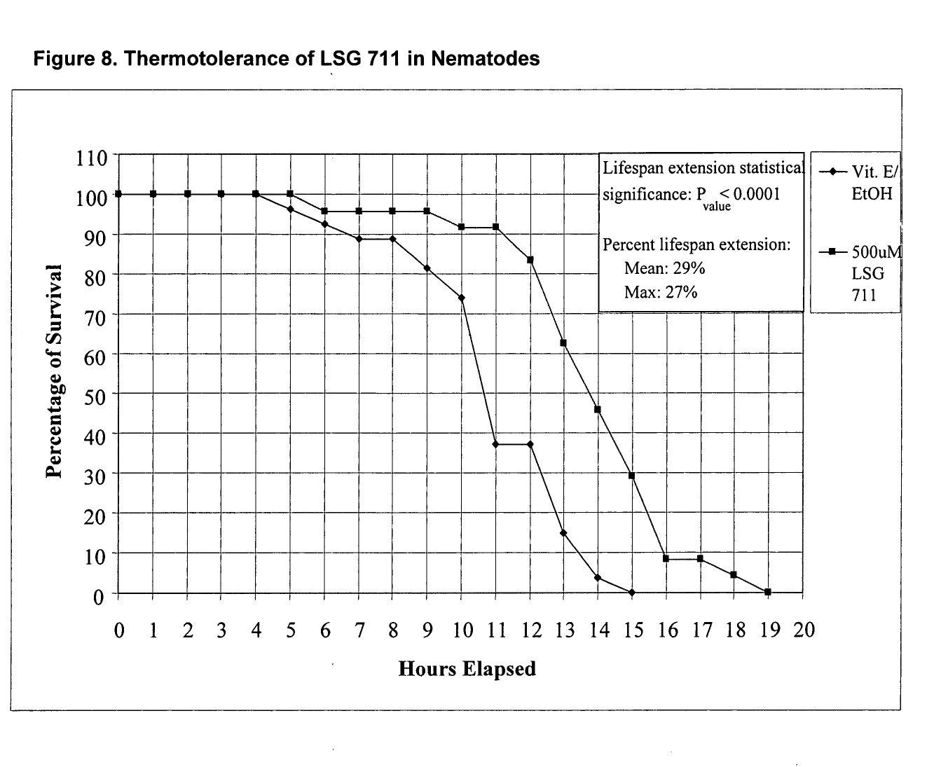

Because GGA has previously been shown to induce the expression of heat shock proteins, the effect of GGA in promoting thermotolerance was tested. The thermotolerance experiments were performed by workers at the Buck Institute on Aging in Novato, California under contract with the applicant. The workers at the Buck Institute performed these experiments under the direction and control of the applicant. The method used is that described in Sampayo et al., Aging Cell, 2: 319-326 (2003). In brief, 50 μl of the test compound dilution was added to a 10 ml week old agar plate spotted with 200 μl of concentrated E. coli OP50. The plates were set aside for 2-5 hours to allow the compounds to disperse. Age synchronous worm cultures of wide type (N2 strain) worms were prepared by hypochroride treatment of fertile adults and grown to three days of age at 20° C. The worms were washed off, resuspended in a small volume of S-basal and approximately 1000 worms were spotted onto each compound plate (˜1000 worms per plate) and the plates were incubated at 20° C. for 24 hours. Thermotolerance was measured using a Fluorskan Ascent fluorometer (Thermo Labsystems, MA). The fluorometer temperature was set to 35 oC and the fluorescence was measured in each well every 30 minutes over a 20-24 hour period, with a 20 millisecond integration time for each well. For SYTOX green fluorescence, the excitation wavelength was set to 485 nm and the emission wavelength at 538 nm. Analysis of individual fluorescence curves to determine the time of death was performed using the Fluoroskan Ascent software. Differences in thermotolerance were assessed using the Mantel-Haenszel Logrank test as implemented in Prism (GraphPad Software Inc.). Kaplan-Meier survival curves were generated using Prism survival analysis. The results using GGA (LSG 711) are shown in FIG. 8. The use of 500 μM GGA resulted in a 29% mean and 27% maximum (Pᵥₐₗᵤₑ<0.0001) increase in lifespan extension in this assay. C. elegans were maintained and handled as described above.

### EXAMPLE 6

Studies to determine the effect of an additional GGA analogue on lifespan were also performed. The class IIIa compound where R₂ is acetone (denoted LSG 712) was used and has the structure shown below.

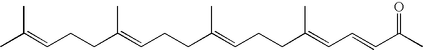

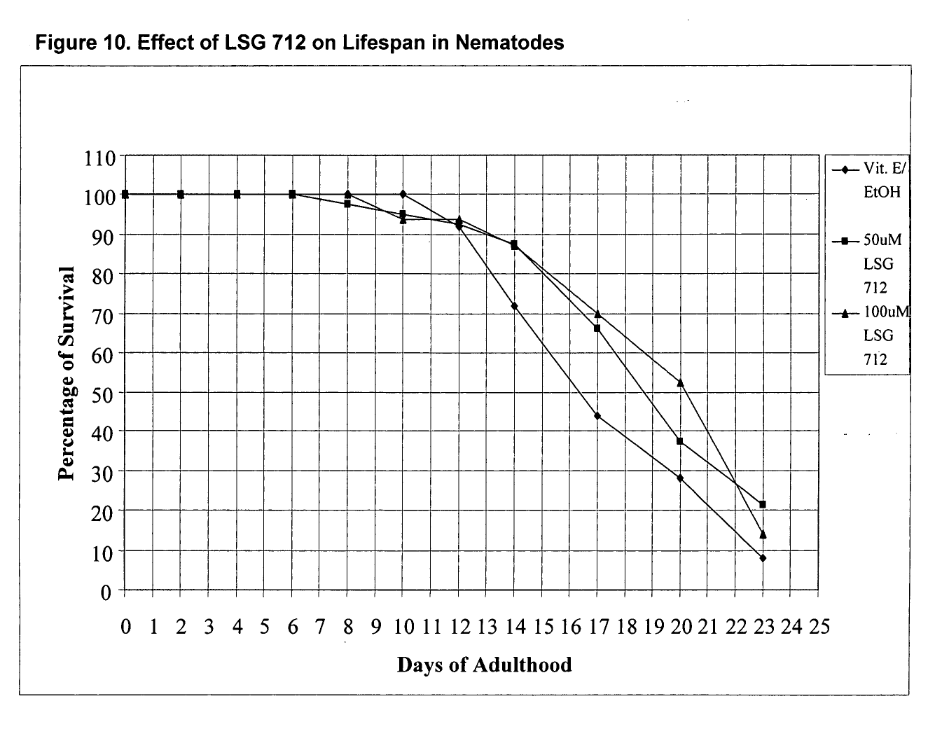

The methods described above for the testing of GGA were employed in the testing of the class IIIa compound, LSG 712. The results of these studies using 50 and 100 μM LSG 712 are shown in FIG. 10. The data in FIG. 10 shows that 50 μM LSG 712 produces a 12% mean percent extension in lifespan (Pᵥₐₗᵤₑ=0.1794). 100 μM LSG 712 produces an 12% mean percent extension in lifespan (Pᵥₐₗᵤₑ=0.055).

### EXAMPLE 7

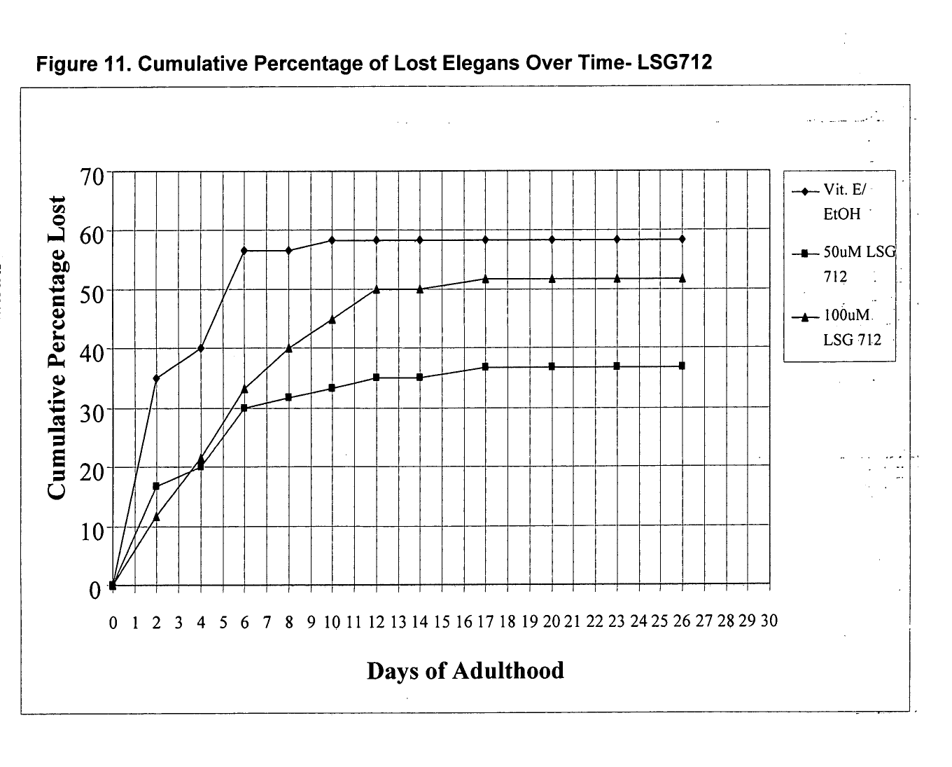

Lifespan extension in the presence of the class IIIa compound where R₂ is acetone (LSG 712) was also measured in C. elegans using the cumulative percent loss of C. elegans over time as an assay. The methods described above for the testing of GGA were employed in the testing of LSG 712. The results of using 50 and 100 μM LSG 712 in this type of assay are shown in FIG. 11. The use of 50 μM LSG 712 resulted in a 25% maximum and 21% mean percent increase in protection (Pᵥₐₗᵤₑ<0.0001). The use of 100 μM LSG 712 resulted in a 20% maximum and 12% mean percent increase in protection (Pᵥₐₗᵤₑ<0.0001).

### EXAMPLE 8

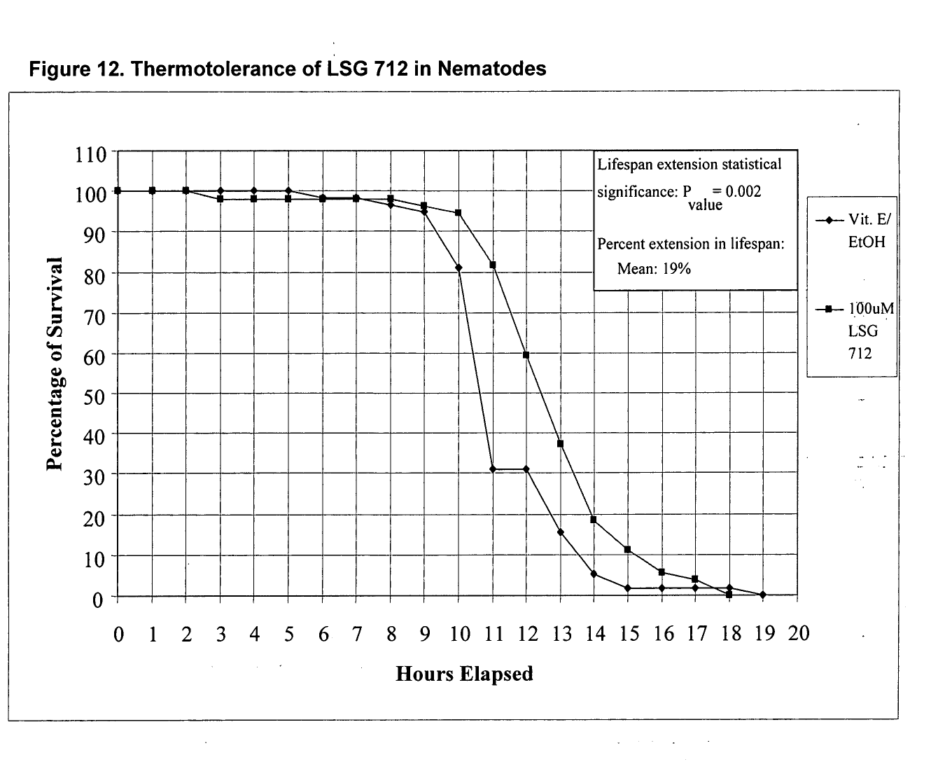

The effect of a GGA analogue promoting thermotolerance was tested. The thermotolerance experiments were performed by workers at the Buck Institute on Aging in Novato, California under contract with the applicant. The workers at the Buck Institute performed these experiments under the direction and control of the applicant. The methods described above for the testing of GGA were employed in the testing of a class IIIa compound where R₂ is acetone (LSG 712). The results using LSG 712 are shown in FIG. 12. The use of 100 μM LSG 712 resulted in a 19% mean (Pᵥₐₗᵤₑ<0.002) increase in lifespan extension in this assay.

### EXAMPLE 9

The effect of LSG 712 on the age dependent accumulation of lipofuscin pigment was measured in C. elegans after treatment of worms with various concentrations of the drug. Synchronized eggs were placed on plates containing no drug and various concentrations of LSG 712 and allowed to hatch. On day 16, the worms were washed in 9M buffer and placed on a microscope slide. The amount of lipofuscin pigment was measured using a confocal microscope measuring fluorescence at 488 nm. A 50% reduction in the amount of lipofuscin pigment was observed in worms that had been treated with LSG 712 when compared with non-treated control worms.

### EXAMPLE 10

The effect of GGA and GGA analogues in mediating resistance to oxidative stress can be measured by treating C. elegans with agents that produce oxidative stress such as paraquat and treating with various concentrations of GGA or GGA analogues. Exposure of worms to paraquat would commence at an age of about 5 days using four different concentrations of paraquat. Protocols for treating worms with paraquat may be found in Sampayo et al., Aging Cell, 2: 319-326 (2003). Percentage survival in the presence of GGA or GGA analogues would be then be measured over time as in Example 1.

### EXAMPLE 11

The effect of GGA and GGA analogues on various markers of oxidative stress can be tested. C. elegans can be maintained in various concentrations of GGA as described in Example 1. At different time points, worms can be harvested and then homogenized to generate extracts that can be used for enzymatic assays or RNA or protein extract preparation. The extracts can be assayed for oxidative damage such as lipid peroxidation or protein carbonyl oxidation. RNA can be used to generate northern blots that can be probed for the expression of various oxidative stress markers, such as superoxide dismutase, catalase, glutathione peroxidase, thioredoxin, or heat shock protein (HSP) 70. Alternatively, western blots can be prepared from the protein extracts and probed with antibodies to various oxidative stress markers, such as superoxide dismutase, catalase, glutathione peroxidase, thioredoxin, or heat shock protein (HSP) 70.

### EXAMPLE 12

The effect of GGA and GGA analogues on maintaining levels of physical activity as a function of age can be measured. As described in Example 1, C. elegans can be maintained in various concentrations of GGA or GGA analogues, and the level of activity of the worms can be monitored over increasing age.

### EXAMPLE 13

The effectiveness of GGA and GGA analogues at modifying longevity parameters when first administered at different stages of life can be determined. For this experiment, worms are maintained as described in Example 1. GGA or GGA analogues can then be applied to the worms at 3 days, 6 days, 12 days, and 20 days of age and the drug maintained through the remaining lifespan of the worms. The effect of GGA or GGA analogues on the lifespan, life expectancy, accumulation of lipofuscin, protection from agents that cause oxidative stress such as paraquat, the expression and activity of various markers of oxidative stress, and physical activity levels of worms can be then be measured as described in Examples 1-6.

### EXAMPLE 14

The additive effect of GGA and GGA analogues on the life extension observed in mutants of C. elegans that show extended lifespan can also be tested. The AGE-1 mutant of C. elegans, which has a lifespan of approximately 55 days as compared to approximately 25 days for wild type worms, can be used. The mechanism underlying the lifespan extension in the AGE-1 mutant is thought to be due to reduction in oxidative stress. The C. elegans DAF-2 mutant has a lifespan of approximately 65 days. The mechanism behind the lifespan extension in the DAF-2 mutant is thought to be due to a decrease in the insulin/IGF-1 receptor activity. The studies described in Examples 1-7 can be conducted on these worms to determine if GGA or GGA analogues will provide additional lifespan extension to these already long lived mutants. These studies can indicate whether GGA and GGA analogues have a mechanism of action separate from the mechanisms underlying the AGE-1 and DAF-2 mutants. The absence of additional lifespan extension in these mutants would indicate that a mechanism of action is common to the mutants and GGA or GGA analogues. The ability of GGA or GGA analogues to provide additional lifespan extension in the mutant strains would indicate that separate mechanisms of action were involved.

1. A compound of the formula: 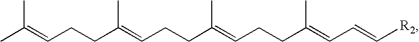
wherein the compound is all-trans or a 5-cis form or a mixture of these two isomers and R₂ is selected from the group consisting of: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearol 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl and 24) 2-hydroxypropyl.

2. The compound of claim 1 wherein the compound is

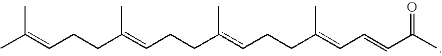

3. A method to reduce oxidative stress in an individual comprising administering an effective amount of a compound of claim 1 to an individual.

4. The method of claim 3, wherein the reduction in oxidative stress extends the lifespan of the individual.

5. The method of claim 3 wherein the compound is administered orally, via gavage, intraperitoneally, or subcutaneously.

6. A method of treating, ameliorating, or preventing a disease resulting from oxidative stress in an individual comprising administering an effective amount of a compound of claim 1 to an individual.

7. The method of claim 6 wherein the disease is selected from the group consisting of inflammatory injury, immune injury, glomerulonephritis, autoimmune diseases, rheumatoid arthritis, hepatitis, chronic inflammatory diseases, ischaemia-reflow diseases, stroke, inflamed rheumatoid joint, ischaemia-reperfusion disorders, iron overload in tissue and plasma, idiopathic haemochromatosis, alcohol-related iron overload, disorders resulting from cancer chemotherapy and radiotherapy, disorders of premature ageing, cancer, lead poisoning, malaria, sickle cell anaemia, respiratory tract disorders, disorders caused by cigarette smoke, emphysema, ARDS (Adult Respiratory Syndrome), atherosclerosis, cardiac iron overload, cardiac ischaemia-reoxygenation disorders, Alzheimer's disease, Parkinson's disease, cataract, retinopathy, UV radiation damage, contact dermatitis, diabetes, diabetic retinopathy, peripheral neuropathy, type II or non-insulin-dependent diabetes, juvenile onset diabetes, and cystic fibrosis.

8. The method of claim 6 wherein the disease is a nervous system disorder.

9. The method of claim 8 wherein the disease is Alzheimer's disease.

10. The method of claim 6 wherein the disease is diabetes.

11. The method of claim 10 wherein the diabetes is type II diabetes.

12. A compound selected from the group consisting of: 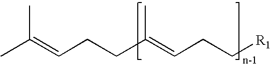
wherein n is 8 or 9 and R1 is selected from the group consisting of 1) acetone, 2) ethyl, 3) hydroxyalkyl 4) amine 5) hydroxyl 6) pivaloyl 7)formyl 8) propionyl 9) butryl ) 10) valeryl 11) isovaleryl 12) stearoyl 13) myristoyl 14) palmitoyl 15) oleoyl 16) benzoyl 17) lauroyl, 18) 2-oxopropyl 19) 2-oxopentyl 20) 3-methyl-2-oxobutyl 21) amino 22) carbethoxyl amino 23) 2-hydroxypropyl 24) retinol acetate; 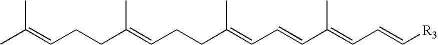
wherein the compound is all-trans or a 5-cis form or a mixture of these two isomers and R₃ is selected from the group consisting of: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl and 24) 2-hydroxypropyl; 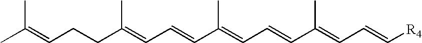
wherein the compound is all-trans or a 5-cis form or a mixture of these two isomers and R₄ is selected from the group consisting of: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl and 24) 2-hydroxypropyl; 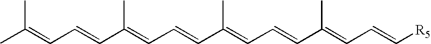
wherein the compound is all-trans or a 5-cis form or mixture of these two isomers and R₅ is selected from the group consisting of: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) and 2-hydroxypropyl; 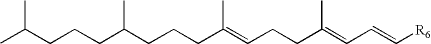
wherein the compound is all-trans or a 5-cis form or a mixture of these two isomers and R₆ is selected from the group consisting of: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) and 2-hydroxypropyl; 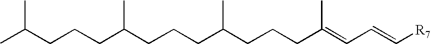
wherein the compound is all-trans or a 5-cis form or a mixture of these two isomers and R₇ is selected from the group consisting of: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) and 2-hydroxypropyl; 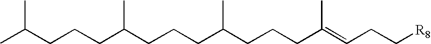
wherein the compound is all-trans or a 5-cis form or a mixture of these two isomers and R₈ is selected from the group consisting of: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) and 2-hydroxypropyl; 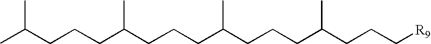
wherein R₉ is selected from the group consisting of: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl and 24) 2-hydroxypropyl; 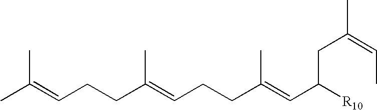
wherein R₁₀ is selected from the group consisting of: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl and 24) 2-hydroxypropyl; 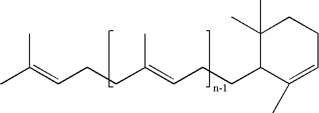
wherein n is 2, 3, or 4 and the repeating units can be in any reduced or oxidized form; 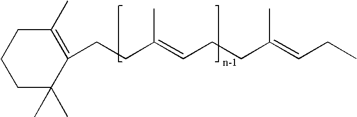
wherein n is 2, 3, or 4 and the repeating units can be in any reduced or oxidized form; 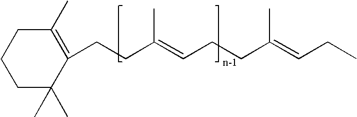
wherein n is 2, 3, or 4 and the repeating units can be in any reduced or oxidized form; 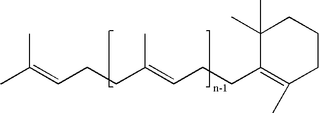
wherein n is 2, 3, or 4 and the repeating units can be in any reduced or oxidized form; 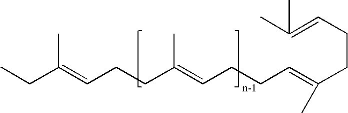
wherein n is 2, 3, or 4 and the repeating units can be in any reduced or oxidized form; 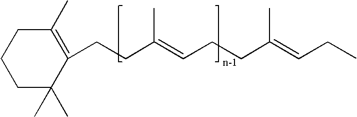
wherein n is 2, 3, or 4 and the repeating units can be in any reduced or oxidized form; 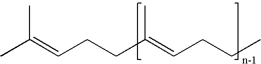
wherein n is 2, 3, or 4 and the repeating units are in any reduced or oxidized form; 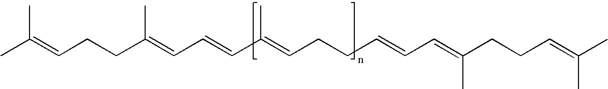
wherein n=2, 3, 4, 5, 6, 8, or 9 and the repeating units are in any reduced or oxidized form; 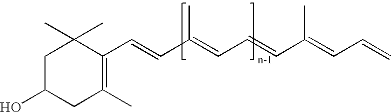
wherein n is 2, 3, or 4 and the repeating units are in any reduced or oxidized form; 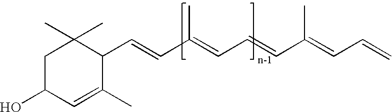
wherein n is 2, 3, or 4 and the repeating units are in any reduced or oxidized form; 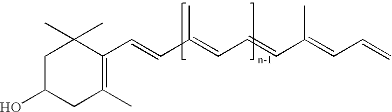
wherein n is 2, 3, or 4 and the repeating units are in any reduced or oxidized form; 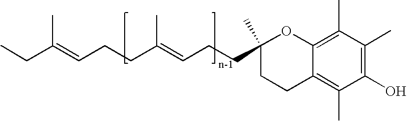
wherein n is 2, 3, or 4 and the repeating units are in any reduced or oxidized form; 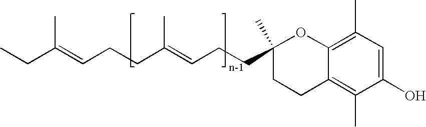
wherein n is 2, 3, or 4 and the repeating units are in any reduced or oxidized form; 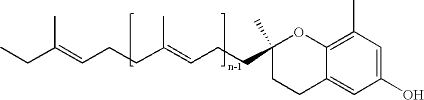
wherein n is 2, 3, or 4 and the repeating units are in any reduced or oxidized form; 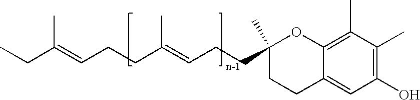
wherein n is 2, 3, or 4 and the repeating units are in any reduced or oxidized form; 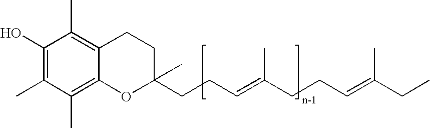
wherein n is 1, 2, 4, 5, 6, 8, or 9 and the repeating units are in any reduced or oxidized form; 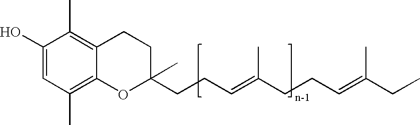
wherein n is 1, 2, 4, 5, 6, 8, or 9 and the repeating units are in any reduced or oxidized form; 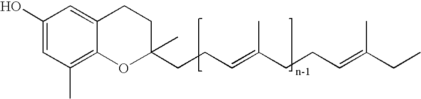
wherein n is 1, 2, 4, 5, 6, 8, or 9 and the repeating units are in any reduced or oxidized form; 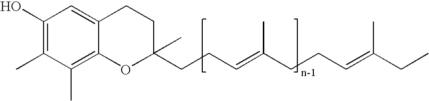
wherein n is 1, 2, 4, 5, 6, 8, or 9 and the repeating units are in any reduced or oxidized form; 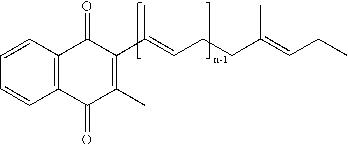
wherein n is 2, 3, or 4 and the repeating units are in any reduced or oxidized form; 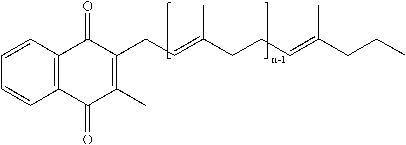
wherein n is 2, 3, or 4 and the repeating units are in any reduced or oxidized form; 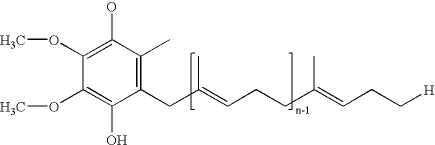
wherein n is 2, 3, 4, 5, 6, 8, or 9; 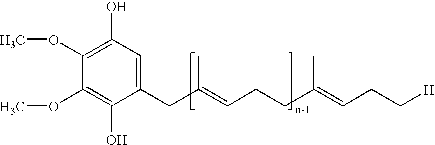
wherein n is 2, 3, 4, 5, 6, 8, or 9; 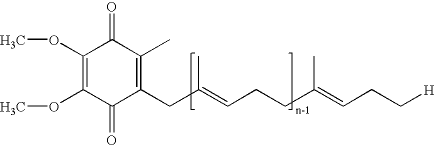
wherein n is 2, 3, 4, 5, 6, 8, or 9; 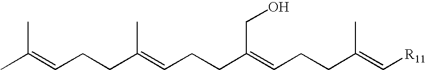
wherein the compound is all-trans or a 5-cis form or a mixture of these two isomers and R₁₁ is selected from the group consisting of: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) and 2-hydroxypropyl; 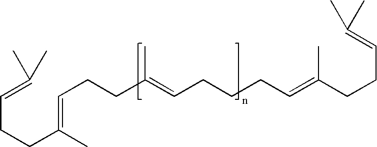
wherein n is 2, 3, or 4 and the repeating units are in any reduced or oxidized form; 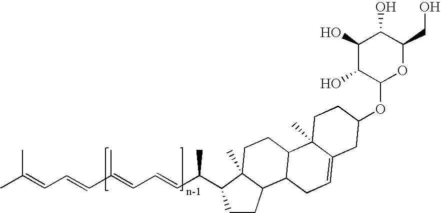
wherein n is 1, 2, 3, or 4 and the repeating units are in any reduced or oxidized form; 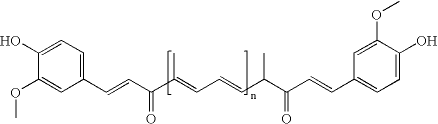
wherein n is 1, 2, 3, or 4 and the repeating units are in any reduced or oxidized form; 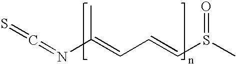
wherein n is 1, 2, 3, or 4 and the repeating units are in any reduced or oxidized form; 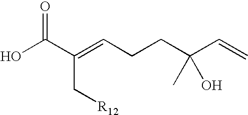
wherein R₁₂ is hydrogen or a hydroxyl group; 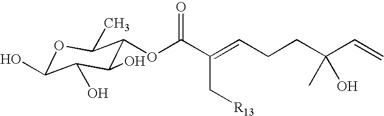
wherein R₁₃ is hydrogen or a hydroxyl group; 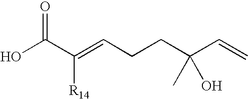
wherein R₁₄ is hydrogen or a hydroxyl group; 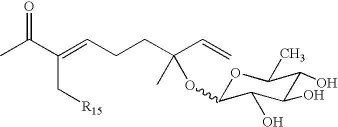
wherein R₁₅ is hydrogen or a hydroxyl group; 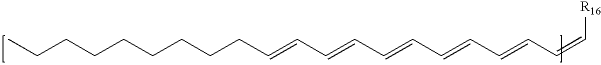
wherein the compound contains 0, 1, 2, 3, 4, 5, 6, 7, or 8 methyl groups and R,₆ is selected from the group consisting of: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) and 2-hydroxypropyl; 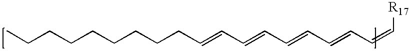
wherein the compound contains 0, 1, 2, 3, 4, 5, 6, 7, or 8 methyl groups and RI₇ is selected from the group consisting of: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) and 2-hydroxypropyl; 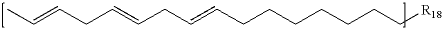
wherein the compound contains 0, 1, 2, 3, 4, 5, 6, 7, or 8 methyl groups and R₁₈ is selected from the group consisting of: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) and 2-hydroxypropyl; 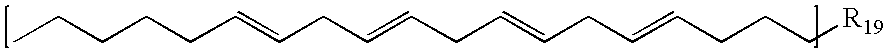
wherein the compound contains 0, 1, 2, 3, 4, 5, 6, 7, or 8 methyl groups and R₁₉ is selected from the group consisting of: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) and 2-hydroxypropyl; 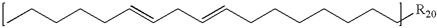
wherein the compound contains 0, 1, 2, 3, 4, 5, 6, 7, or 8 methyl groups and R₂₀ is selected from the group consisting of: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) and 2-hydroxypropyl; 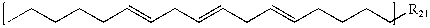
wherein the compound contains 0, 1, 2, 3, 4, 5, 6, 7, or 8 methyl groups and R₂₁ is selected from the group consisting of: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) and 2-hydroxypropyl; 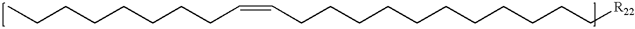
wherein the compound contains 0, 1, 2, 3, 4, 5, 6, 7, or 8 methyl groups and R₂₂ is selected from the group consisting of: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) and 2-hydroxypropyl; 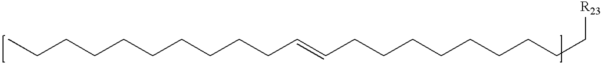
wherein the compound contains 0, 1, 2, 3, 4, 5, 6, 7, or 8 methyl groups and R₂₃ is selected from the group consisting of: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) and 2-hydroxypropyl; and 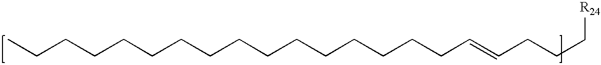
wherein the compound contains 0, 1, 2, 3, 4, 5, 6, 7, or 8 methyl groups and R₂₄ is selected from the group consisting of: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) and 2-hydroxypropyl.

13. (canceled)

14. The compound of claim 12 wherein the compound is: 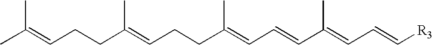
wherein the compound is all-trans or a 5-cis form or a mixture of these two isomers and R₃ is selected from the group consisting of: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl and 24) 2-hydroxypropyl.

15. The compound of claim 12 wherein the compound is: 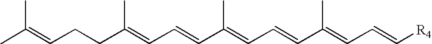
wherein the compound is all-trans or a 5-cis form or a mixture of these two isomers and R₄ is selected from the group consisting of: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl and 24) 2-hydroxypropyl.

16. The compound of claim 12 wherein the compound is: 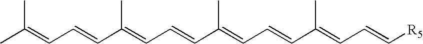
wherein the compound is all-trans or a 5-cis form or mixture of these two isomers and R₅ is selected from the group consisting of: 1) acetone, 2) ethyl, 3) hydroxyalkyl 4) amine 5) hydroxyl 6)formyl 7) propionyl 8) butryl ) 9) 2-oxopropyl 10) 2-oxopentyl 11) retinol acetate 12) stearoyl 12) myristoyl 13) valeryl 14) isovaleryl 15) pivaloyl 16) palmitoyl 17) oleoyl 18) benzoyl 19) lauroyl 20) stearoyl 21) amino 22) carbethoxyl amino 23) 3-methyl-2-oxobutyl 24) and 2-hydroxypropyl.

17-63. (canceled)

64. A method to reduce oxidative stress in an individual comprising administering an effective amount of a compound of claim 12 to an individual.

65. The method of claim 64, wherein the reduction in oxidative stress extends the lifespan of the individual.

66. The method of claim 64 wherein the compound is administered orally, via gavage, intraperitoneally, or subcutaneously.

67. A method of treating, ameliorating, or preventing a disease resulting from oxidative stress in an individual comprising administering an effective amount of a compound of claim 12 to an individual.

68. The method of claim 67 wherein the disease is selected from the group consisting of inflammatory injury, immune injury, glomerulonephritis, autoimmune diseases, rheumatoid arthritis, hepatitis, chronic inflammatory diseases, ischaemia-reflow diseases, stroke, inflamed rheumatoid joint, ischaemia-reperfusion disorders, iron overload in tissue and plasma, idiopathic haemochromatosis, alcohol-related iron overload, disorders resulting from cancer chemotherapy and radiotherapy, disorders of premature ageing, cancer, lead poisoning, malaria, sickle cell anaemia, respiratory tract disorders, disorders caused by cigarette smoke, emphysema, ARDS (Adult Respiratory Syndrome), atherosclerosis, cardiac iron overload, cardiac ischaemia-reoxygenation disorders, Alzheimer's disease, Parkinson's disease, cataract, retinopathy, UV radiation damage, contact dermatitis, diabetes, diabetic retinopathy, peripheral neuropathy, type II or non-insulin-dependent diabetes, juvenile onset diabetes, and cystic fibrosis.

69. The method of claim 67 wherein the disease is a nervous system disorder.

70. The method of claim 69 wherein the disease is Alzheimer's disease.

71. The method of claim 67 wherein the disease is diabetes.

72. The method of claim 71 wherein the diabetes is type II diabetes.

73. A method of treating a disease selected from the group consisting of: inflammatory injury, immune injury, glomerulonephritis, autoimmune diseases, rheumatoid arthritis, hepatitis, chronic inflammatory diseases, ischaemia-reflow diseases, stroke, inflamed rheumatoid joint, ischaemia-reperfusion disorders, iron overload in tissue and plasma, idiopathic haemochromatosis, alcohol-related iron overload, disorders resulting from cancer chemotherapy and radiotherapy, disorders of premature ageing, cancer, lead poisoning, malaria, sickle cell anaemia, respiratory tract disorders, disorders caused by cigarette smoke, emphysema, ARDS (Adult Respiratory Syndrome), atherosclerosis, cardiac iron overload, cardiac ischaemia-reoxygenation disorders, Alzheimer's disease, Parkinson's disease, cataract, retinopathy, UV radiation damage, contact dermatitis, diabetes, diabetic retinopathy, peripheral neuropathy, type II or non-insulin-dependent diabetes, juvenile onset diabetes, and cystic fibrosis, comprising administering a therapeutically effective amount of a compound of claim 12 to an individual.

74. The method of claim 73 wherein the compound is administered orally, via gavage, intraperitoneally, or subcutaneously.

75-77. (canceled)

78. A method of treating a disease selected from the group consisting of: inflammatory and immune injury, glomerulonephritis, autoimmune disease, rheumatoid arthritis, hepatitis, chronic inflammatory disease, stroke, inflamed rheumatoid joint; iron overload, idiopathic haemochromatosis, alcohol-related iron overload, disorders of premature ageing, lead poisoning, malaria, sickle cell anaemia, emphysema, ARDS (Adult Respiratory Syndrome), cardiac iron overload, neuromuscular disorders, cataract, retinopathy, UV radiation damage to skin, contact dermatitis; tumors, diabetes, hyperglycaemia, diabetic retinopathy, peripheral neuropathy, type II or non-insulin-dependent diabetes, juvenile onset diabetes, and cystic fibrosis, comprising administering a therapeutically effective amount of a compound the formula: 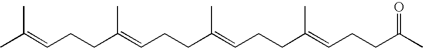
to an individual.
